SEQUENCE LISTING (25 sequences)

SEQ ID NO: 1 - RNA, 26 nt, Homo sapiens
ccccccacugcuaaauuugacugguu

SEQ ID NO: 2 - RNA, 27 nt, Homo sapiens
ccccccacugcuaaauuugacuggcua

SEQ ID NO: 3 - RNA, 27 nt, Homo sapiens
aggggcccgugccuuggaaagcgucgc

SEQ ID NO: 4 - RNA, 29 nt, Homo sapiens
ugagaacuagcuaaacagggucgggcaga

SEQ ID NO: 5 - RNA, 29 nt, Homo sapiens
ugaucaguagugggaucgcgccugugaau

SEQ ID NO: 6 - RNA, 31 nt, Homo sapiens
uugcugugaugacuaucuuaggacaccuuug

SEQ ID NO: 7 - RNA, 27 nt, Homo sapiens
ugggaaugcagcccaaagcggguggua

SEQ ID NO: 8 - RNA, 31 nt, Homo sapiens
gagaggggcccgugccuuggaaagcgucgcg

SEQ ID NO: 9 - RNA, 29 nt, Homo sapiens
ggucagucgguccugagagaugggcgagc

SEQ ID NO: 10 - RNA, 30 nt, Homo sapiens
gggagaugaagaggacagugacugagagac

SEQ ID NO: 11 - RNA, 31 nt, Homo sapiens
ccgccugggaauaccgggugcuguaggcuua

SEQ ID NO: 12 - RNA, 29 nt, Homo sapiens
aucagaccccagaaaagguguugguugau

SEQ ID NO: 13 - RNA, 27 nt, Homo sapiens
gugguggcggcggcggcggaggcggac

SEQ ID NO: 14 - RNA, 32 nt, Homo sapiens
agccugagcaacauagcgagaccccgucucua

SEQ ID NO: 15 - RNA, 32 nt, Homo sapiens
ucccugguggucuagugguuaggauucggcac

SEQ ID NO: 16 - RNA, 32 nt, Homo sapiens
uuggugguucagugguagaauucucgccugcc

SEQ ID NO: 17 - RNA, 30 nt, Homo sapiens
ccccugguggucuagugguuaggauucggc

SEQ ID NO: 18 - RNA, 31 nt, Homo sapiens
uucccugguggucuagugguuaggauucggc

SEQ ID NO: 19 - RNA, 22 nt, Homo sapiens
uauggcacugguagaauucacu

SEQ ID NO: 20 - RNA, 24 nt, Homo sapiens
uuuggcaaugguagaacucacacu

SEQ ID NO: 21 - RNA, 23 nt, Homo sapiens
ugugcaaaucuaugcaaaacuga

SEQ ID NO: 22 - RNA, 22 nt, Homo sapiens
uauugcacuugucccggccugu

SEQ ID NO: 23 - RNA, 23 nt, Homo sapiens
caaagugcuuacagugcagguag

SEQ ID NO: 24 - RNA, 22 nt, Homo sapiens
ucguaccgugaguaauaaugcg

SEQ ID NO: 25 - RNA, 21 nt, Homo sapiens
auugaaaccucuaagagugga

(19) United States
(12) Patent Application Publication
(10) Pub. No.: US 2023/0203487 A1
(43) Pub. Date: Jun. 29, 2023

(54) EXOSOME-DERIVED PIWI-INTERACTING RNA AND METHODS OF USE THEREOF

(71) Applicant: Cedars-Sinai Medical Center, Los Angeles, CA (US)

(72) Inventors: Eduardo Marbán, Santa Monica, CA (US); Alessandra Ciullo, Los Angeles, CA (US); Ahmed G. Ibrahim, Los Angeles, CA (US)

(21) Appl. No.: 17/998,857

(22) PCT Filed: May 17, 2021
(86) PCT No.: PCT/US21/70568

§ 371 Date: Nov. 15, 2022

Related U.S. Application Data
(60) Provisional application No. 63/027,191, filed on May 19, 2020.

(51) Int. Cl. C12N 15/113; A61P 9/10; A61P 11/00; A61K 35/33; A61K 31/7105; A61K 35/28

(52) U.S. Cl. CPC C12N 15/113; A61P 9/10; A61P 11/00; A61K 35/33; A61K 31/7105; A61K 35/28; C12N 2310/14; C12N 2320/35; C12N 2320/32; C12N 2310/141

(57) ABSTRACT

Provided herein are PIWI-interacting RNA (piRNA) derived from therapeutic exosomes, and methods of use thereof to treat a condition requiring tissue repair and/or regeneration. Conditions treated by the exosome-derived piRNAs and/or exosomes carrying the same include, in some embodiments, ischemic injury and/or tissue fibrosis. Also provided are therapeutic compositions comprising exosome-derived piRNA and a pharmaceutically acceptable excipient.

### REFERENCE TO RELATED APPLICATIONS

This application claims priority to U.S. Provisional Application No. 63/027,191, filed May 19, 2020, the entirety of which is incorporated herein by reference.

### STATEMENT REGARDING FEDERALLY SPONSORED R&D

This invention was made with government support under Grant No. R01 HL124074, awarded to Dr. Eduardo Marbán by the National Institutes of Health. The Government has certain rights in the invention.

### REFERENCE TO SEQUENCE LISTING

The present application is being filed along with a Sequence Listing in electronic format. The Sequence Listing is provided as a file entitled SEQLIST_CSMC014WO.txt created on May 16, 2021, which is 3.85 KB in size. The information in the electronic format of the Sequence Listing is incorporated herein by reference in its entirety.

### BACKGROUND

### Field

The present disclosure generally relates to PIWI-interacting RNA (piRNA) derived from exosomes and uses thereof to treat conditions requiring tissue repair and/or regeneration.

piRNAs are a group of small, non-coding RNAs that associate with PIWI proteins, and are known to function in gene silencing retrotransposons and other genetic elements in germ line cells. Cytoplasmic PIWI proteins are small RNA guided nucleases (slicers) that guide endonucleolytic cleavage of transposon targets, while nuclear PIWI proteins assemble silencing complexes on target genomic loci to mediate transcriptional silencing.

Cardiosphere-derived cells (CDCs) are a population of heart-derived progenitors with demonstrated therapeutic efficacy in preclinical and clinical settings. CDCs function by secreting extracellular vesicles (EVs), lipid-bilayer nanoparticles laden with bioactive molecules. Immortalizing CDCs (imCDC) that retain therapeutic potential can be generated and can provide enhanced CDC function through their secreted EVs.

### SUMMARY

Provided herein is an exosome-free and optionally cell-free method of treating ischemic cardiac muscle injury, comprising: identifying a subject having or in need of treating an ischemic cardiac muscle injury; administering to the subject an effective (or therapeutically effective) amount of an exosome-derived PIWI-interacting RNA (piRNA). In several embodiments, the effective (or therapeutically effective) amount comprises from about 80 ng to about 5 mg of the piRNA, to thereby treat the ischemic cardiac muscle injury. Optionally, the exosome-derived piRNA comprises CDC (cardiosphere-derived cells)-derived exosomal piRNA. Optionally, the ischemic cardiac muscle injury comprises ischemic/reperfusion injury. In some embodiments, the ischemic cardiac muscle injury comprises cardiac muscle fibrosis. In some embodiments, the subject has suffered a myocardial infarction. In some embodiments, the effective amount (or therapeutically effective) of exosome-derived piRNA is administered about 10 minutes to about 2 hours after the ischemic cardiac muscle injury.

Also provided is an exosome-free and optionally cell-free method of treating ischemic cardiac muscle injury, comprising: identifying a subject in need of treating an ischemic cardiac muscle injury; and administering to the subject an effective (or therapeutically effective) amount of a piRNA comprising a nucleotide sequence of hsa_piR_016659, to thereby treat the ischemic cardiac muscle injury. Additionally, provided for herein is an exosome-free method of treating muscle injury (for example cardiac muscle injury), comprising administering to a subject an effective (or therapeutically effective) amount of a piRNA (such as hsa_piR_016659), to thereby treat the muscle injury. Optionally, the piRNA consists of the nucleotide sequence of hsa_piR_016659. In some embodiments, the ischemic cardiac muscle injury comprises ischemic/reperfusion injury. In some embodiments, the ischemic cardiac muscle injury comprises cardiac muscle fibrosis. In some embodiments, the subject has suffered a myocardial infarction. In some embodiments, the therapeutically effective amount of the piRNA is administered about 10 minutes to about 2 hours after the ischemic cardiac muscle injury. In some embodiments, the therapeutically effective amount comprises from about 80 ng to about 5 mg of the piRNA. In some embodiments, the piRNA comprises one or more chemically modified nucleotides.

Also provided herein is an exosome-free method of treating a condition requiring tissue repair and/or regeneration, comprising: identifying a subject having a condition requiring tissue repair and/or regeneration; and administering to the subject an effective (or therapeutically effective) amount of exosome-derived PIWI-interacting RNA (piRNA), wherein the effective (or therapeutically effective) amount comprises from about 80 ng to about 5 mg of the piRNA, to thereby treat the condition requiring tissue repair and/or regeneration. Optionally, the condition requiring tissue repair and/or regeneration comprises injury to muscle or lung tissue. Optionally, the muscle tissue comprises skeletal or cardiac muscle. In some embodiments, the condition comprises or is a condition that causes tissue fibrosis. In some embodiments, the condition comprises ischemic cardiac muscle injury or pulmonary fibrosis. In some embodiments, the exosome-derived piRNA comprises fibroblast-derived exosomal piRNA or CDC (cardiosphere-derived cells)-derived exosomal piRNA.

In several embodiments of a method of treating ischemic cardiac muscle injury or treating a condition requiring tissue repair and/or regeneration, the exosome-derived piRNA comprises one or more of: hsa_piR_016659 (SEQ ID NO: 1), hsa_piR_016658 (SEQ ID NO: 2), hsa_piR_001040 (SEQ ID NO: 3), hsa_piR_007424 (SEQ ID NO: 4), hsa_piR_008488 (SEQ ID NO: 5), hsa_piR_018292 (SEQ ID NO: 6), hsa_piR_013624 (SEQ ID NO: 7), hsa_piR_019324 (SEQ ID NO: 8), and hsa_piR_020548 (SEQ ID NO: 9). In some embodiments, the exosome-derived piRNA is hsa_piR_016659, variant thereof and/or fragment thereof.

Also provided herein is a cell-free method of treating a condition requiring tissue repair and/or regeneration, comprising: identifying a subject having a condition requiring tissue repair and/or regeneration; and administering to the subject an effective (or therapeutically effective) amount of exosome-derived PIWI-interacting RNA (piRNA), to thereby treat the condition requiring tissue repair and/or regeneration, wherein the exosome-derived piRNA comprises one or more of hsa_piR_016659, hsa_piR_016658, hsa_piR_001040, hsa_piR_007424, hsa_piR_008488, hsa_piR_018292, hsa_piR_013624, hsa_piR_019324, hsa_piR_020548, piR-20450, piR-16735, piR-01184, piR-20786, piR-00805, piR-04153, piR-18570, piR-16677, and piR-17716. Optionally, administering comprises administering an effective (or therapeutically effective) amount of exosomes, extracellular vesicles or liposomes comprising the exosome-derived piRNA, wherein the exosomes, extracellular vesicles or liposomes are enriched for the exosome-derived piRNA. In some embodiments, the effective (or therapeutically effective) amount comprises from about 80 ng to about 5 mg of the exosome-derived piRNA. Depending on the embodiment, the condition requiring tissue repair and/or regeneration comprises injury to muscle and/or lung tissue. In some embodiments, the condition comprises or is a condition that causes tissue fibrosis. In some embodiments, the exosome-derived piRNA comprises fibroblast-derived exosomal piRNA or CDC (cardiosphere-derived cells)-derived exosomal piRNA.

In some embodiments, the piRNA, e.g., the effective (or therapeutically effective) amount of exo some-derived piRNA, is administered intravenously, intra-arterially, intramuscularly, intracardially, intramyocardially or intratracheally.

In some embodiments, the exosome-derived piRNA comprises one or more chemically modified nucleotides.

Also provided herein is a cell-free method of treating pulmonary fibrosis, comprising: identifying a subject having pulmonary fibrosis; and administering to the subject an effective (or therapeutically effective) amount of therapeutic exosomes, exosome-derived miRNA, and/or exosome-derived PIWI-interacting RNA (piRNA), to thereby treat the pulmonary fibrosis, wherein the exosomes are derived from engineered fibroblasts. Optionally, the effective (or therapeutically effective) amount of therapeutic exosomes, exosome-derived miRNA, and/or exosome-derived piRNA is administered intratracheally. In some embodiments, the effective (or therapeutically effective) amount of the therapeutic exosomes comprises from about 10⁶ to about 10¹² particles.

Also provided herein is a method of regulating tissue repair, comprising contacting a population of transdifferentiating fibroblasts with an effective (or therapeutically effective) amount of exosomes, exosome-derived miRNA, and/or exosome-derived PIWI-interacting RNA (piRNA), to thereby suppress transdifferentiation of the fibroblasts into myofibroblasts, wherein the exosomes are derived from engineered fibroblasts. Optionally, the effective (or therapeutically effective) amount of exosomes comprises about 10⁶ to about 10¹² particles. In some embodiments, the transdifferentiation is TGFβ-mediated transdifferentiation. In some embodiments, the contacting is done in vitro. Optionally, the effective (or therapeutically effective) amount of piRNA comprises from about 1 nM to about 200 nM.

In some embodiments, the contacting comprises administering the exosomes to a subject. In some embodiments, the fibroblasts are lung fibroblasts. Optionally, contacting comprises administering the exosomes, exosome-derived miRNA, and/or exosome-derived piRNA to a subject intratracheally. In some embodiments, the subject has pulmonary fibrosis.

In some embodiments, the exosome-derived miRNA comprises one or more of miR-183-5p (SEQ ID NO: 19), miR-182-5p (SEQ ID NO: 20), miR-19a-3p (SEQ ID NO: 21), miR-92a-3p (SEQ ID NO: 22), miR-17-5p (SEQ ID NO: 23), miR-126-3p (SEQ ID NO: 24), and miR-510-3p (SEQ ID NO: 25). In some embodiments, the exosome-derived piRNA comprises one or more of piR-20450 (SEQ ID NO: 10), piR-20548 (SEQ ID NO: 9), piR-16735 (SEQ ID NO: 11), piR-01184 (SEQ ID NO: 12, piR-20786 (SEQ ID NO: 13), piR-00805 (SEQ ID NO: 14), piR-04153 (SEQ ID NO: 15), piR-18570 (SEQ ID NO: 16), piR-16677 (SEQ ID NO: 17), and piR-17716 (SEQ ID NO: 18), variant thereof and/or fragment thereof.

In some embodiments, the method includes isolating the piRNA, e.g., exosome-derived piRNA, from therapeutic exosomes. Optionally, the therapeutic exosomes are CDC-derived exosomes or fibroblast-derived exosomes.

In some embodiments, the method includes isolating the therapeutic exosomes from a population of therapeutic cells. Optionally, the method includes generating the population of therapeutic cells from non-therapeutic cells. In some embodiments, the non-therapeutic cells comprise fibroblasts or CDCs. Optionally, the CDCs are immortalized CDCs.

In some embodiments, the therapeutic cells are allogeneic. In several embodiments, the therapeutic cells are administered prior to, concurrently with, or after the piRNA is administered.

In some embodiments, the effective (or therapeutically effective) amount of exosome-derived piRNA is from about 80 ng to about 500 μg. In some embodiments, the effective (or therapeutically effective) amount of exosome-derived piRNA is from about 100 ng to about 10 μg (e.g., about 100 ng, about 200 ng, about 300 ng, about 400 ng, about 500 ng, about 600 ng, about 700 ng, about 800 ng, about 900 ng, about 1 μg, about 2 μg, about 3 μg, about 4 μg, about 5 μg, about 6 μg, about 7 μg, about 8 μg, about 9 μg, about 10 μg, and any amount therebetween. In some embodiments, the effective (or therapeutically effective) amount of the piRNA, e.g., exosome-derived piRNA, is an amount having a therapeutic effect equivalent to a therapeutic effect of administering from about 10⁹ to about 10¹² immortalized CDC-derived exosomes.

Also provided herein is a use of exosome-derived PIWI-interacting RNA (piRNA) to treat ischemic cardiac injury in a subject in need thereof. Also provided is a use of exosome-derived PIWI-interacting RNA (piRNA) for the preparation of a medicament to treat ischemic cardiac injury in a subject in need thereof. Provided herein is a use of therapeutic exosomes and/or exosome-derived PIWI-interacting RNA (piRNA) to treat pulmonary fibrosis in a subject in need thereof. Also provided herein is a use of therapeutic exosomes and/or exosome-derived PIWI-interacting RNA (piRNA) for the preparation of a medicament to treat pulmonary fibrosis in a subject in need thereof.

Also provided herein is an exosome-free therapeutic composition for treatment of a condition requiring tissue repair and/or regeneration, comprising: one or more exosome-derived piRNAs selected from hsa_piR_016659, hsa_piR_016658, hsa_piR_001040, hsa_piR_007424, hsa_piR_008488, hsa_piR_018292, hsa_piR_013624, hsa_piR_019324, and hsa_piR_020548; and a pharmaceutically acceptable excipient. Optionally, the composition consists essentially of the one or more exosome-derived piRNAs and the pharmaceutically acceptable excipient. In some embodiments, the one or more exosome-derived piRNAs is hsa_piR_016659. In some embodiments, the condition comprises or is a condition that causes tissue fibrosis. In some embodiments, the condition comprises ischemic cardiac muscle injury or pulmonary fibrosis. In some embodiments, the one or more exosome-derived piRNAs comprises fibroblast-derived exosomal piRNA or CDC (cardiosphere-derived cells)-derived exosomal piRNA.

### BRIEF DESCRIPTION OF THE DRAWINGS

FIG. 1A shows a schematic protocol for isolation of imCDC (immortalized cardiosphere-derived cell)-derived exosomes (IMEX).

FIG. 1B shows IMEX piRNA (PIWI-interacting RNA) in primary CDCs (pCDCs), imCDCs, and extracellular vesicles/exosomes (EVs) from pCDCs (pCDC-EVs) and imCDCs (imCDC-EVs).

FIG. 1C shows detection by qPCR of ImEV-piRNA in imCDC-EVs at different concentrations.

FIG. 2A shows schematic protocol of an in vivo Ischemia/Reperfusion (I/R) model.

FIG. 2B shows triphenyltetrazolium chloride (TTC) staining of the heart at 48 hrs after I/R.

FIGS. 3A and 3B show cardiac Troponin I levels (ng/ml) at 24 hrs and 48 hrs after I/R.

FIGS. 4A and 4B illustrate the effect of ImEV-piRNA on the percentage of monocytes in peripheral blood after I/R. FIG. 4A shows the percentage of monocytes at 24 hrs and 48 hrs after I/R. FIG. 4B shows the change in percentage of monocytes from 24 hrs to 48 hrs after I/R.

FIGS. 5A and 5B show the percentage of monocytes in peripheral blood at 24 hrs and 48 hrs after I/R.

FIGS. 6A-6C illustrate an in vitro assessment of proliferative activity of naïve (M0) BMDM (bone marrow-derived macrophage). FIG. 6A shows images of BMDM-derived M0 treated as indicated for 24 hrs. FIG. 6B shows CCK-8 assay detecting metabolic activity of cells at 8 hrs and 24 hrs (FC (fold change) vs Vehicle). FIG. 6C shows BrDu positive cells at 24 hrs (FC vs Vehicle).

FIGS. 7A and 7B illustrates an in vitro assessment of migration of BMDM-derived M0 after overnight treatment. FIG. 7A shows images of BMDM-derived M0 in polycarbonate inserts stained with Crystal violet after overnight treatment with the indicated conditions. FIG. 7B shows calculation of migration of BMDM-derived M0.

FIGS. 8A-8D illustrates that sequencing of ASTEX (extracellular vesicles/exosomes from Activated-Specialized Tissue Effector Cells (ASTECs)) reveals several anti-fibrotic mediators. FIGS. 8A and 8B show differential gene expression of miRs in ASTEX compared to the EVs in unmodified normal human dermal skin fibroblasts. FIG. 8C shows QPCR validation of notable anti-fibrotic miRs. FIG. 8D shows enrichment and abundance of Piwi RNA (piRNA) species in ASTEX compared to fibroblast EVs.

FIGS. 9A-9C illustrate a dose tolerance study for intratracheal administration of ASTEX. FIG. 9A shows a study scheme for the dose tolerance study. ASTEX are well tolerated in the lungs in healthy animals as shown by retention of animal weight (FIG. 9B) and lack of edema (lung weight to body weight ratio) (FIG. 9C).

FIGS. 10A-10D shows ASTEX are well tolerated in the lungs in healthy animals as shown by absence of fibrosis (hydropxyproline, FIG. 10A), Ashcroft Score (FIG. 10B), H&E staining showing lack of infiltrating leukocytes (FIG. 10C) and Masson's trichrome staining in alveolar tissue (FIG. 10D).

FIGS. 11A-11C illustrate intratracheally instilled ASTEX improve survival and attenuate lung fibrosis in mouse bleomycin model. FIG. 11A shows the study scheme for the animal study. FIG. 11B shows a Kaplan-Meir plot showing increased survival of animals instilled with ASTEX compared to vehicle-treated injured animals. FIG. 11C shows reduced fibrosis in the lung as seen by decreased hydroxyproline in lung tissue.

FIGS. 12A-12D illustrate ASTEX can reduce lung fibroblast transdifferentiation in vitro. FIG. 12A shows the study scheme of the in vitro study. Attenuated levels of alpha smooth muscle expression in ASTEX-treated, TGFb (injury)-exposed human lung fibroblasts were observed by flow cytometry (FIG. 12B) and western blot (FIGS. 12C and 12D).

FIG. 13 shows a flow chart of a non-limiting example of a method of treating ischemic cardiac muscle injury, according to embodiments of the present disclosure.

FIG. 14 shows a flow chart of a non-limiting example of a method of treating a condition requiring tissue repair and/or regeneration, according to embodiments of the present disclosure.

FIG. 15 shows a flow chart of a non-limiting example of a method of treating pulmonary fibrosis, according to embodiments of the present disclosure.

FIG. 16 shows the nucleotide sequences of human piRNA sequences hsa_piR_016659, hsa_piR_016658, hsa_piR_001040, hsa_piR_007424, hsa_piR_008488, hsa_piR_018292, hsa_piR_013624, hsa_piR_019324, hsa_piR_020548, piR-20450, piR-16735, piR-01184, piR-20786, piR-00805, piR-04153, piR-18570, piR-16677, and piR-17716.

FIGS. 17A and 17B are a collection of graphs showing imCDC-EV piRNA shuttling between cytoplasm and nucleus in primary macrophages.

FIG. 18A and FIG. 18B are a collection of graphs showing imCDC-EV piRNA treatment increasing global methylation in primary macrophages.

FIG. 19 is a schematic diagram summarizing the in vivo and in vitro effects of imCDC-EV and/or imCDC-EV piRNA.

### DETAILED DESCRIPTION

As disclosed herein, the therapeutic effect of exosomes and extracellular vesicles (EVs) produced by CDCs can be attributed to one or more bioactive payload molecules of the exosomes or EVs. For example, imCDCs can show a different RNA content (miRNA, mRNA, rRNA, tRNA and piRNA) compared to primary CDCs. In particular, Piwi RNAs (piRNAs), small RNAs bound by Piwi proteins, are important regulators of both the epigenome and transcriptome. ImCDC-EVs (imEV-Pi) can be highly enriched in piRNA.

Provided herein are methods of treating a condition requiring tissue repair and/or regeneration by administering exosome-derived PIWI-interacting RNA (piRNA) to a subject in need thereof. As disclosed herein, exosomes/EVs produced by therapeutic cells, e.g., immortalized cardiosphere-derived cells (imCDCs) and engineered fibroblasts, can contain bioactive biomolecules, such as piRNA and miRNA, which can mediate the therapeutic effects of the exosomes and/or cells. In some embodiments, administering exosome-derived piRNA to a subject suffering from a condition, e.g., ischemic injury, fibrosis, etc., can treat the condition. In some embodiments, administering therapeutic exosomes containing piRNA to a subject suffering from a condition, e.g., ischemic injury, fibrosis, etc., can treat the condition.

As used herein, “exosome” has its ordinary meaning as understood by one of ordinary skill in the art and in view of the present disclosure. Exosomes may also include microvesicles, epididimosomes, argosomes, exosome-like vesicles, microparticles, promininosomes, prostasomes, dexosomes, texosomes, dex, tex, archeosomes and oncosomes. Exosomes and extracellular vesicles (EVs) are used interchangeably herein, unless indicated otherwise. Unless otherwise indicated herein, each of the foregoing terms shall also be understood to include engineered high-potency varieties of each type of membrane-bound vesicle.

“PIWI-interacting RNA” and “piRNA” are used interchangeably herein to refer to small, non-coding RNA of about 24 to about 32 nucleotides, e.g., about 26 to about 32 nucleotides long. Endogenous piRNA can associate with PIWI proteins (e.g., Piwi, Argonaute (such as Ago3), and Aubergine). Endogenous piRNA can be complementary to host transposable elements.

Wnt signaling pathways are a group of signal transduction pathways which begin with proteins that pass signals into a cell through cell surface receptors. Canonical and non-canonical Wnt signaling pathways are known. Both canonical and noncanonical Wnt signaling pathways are activated by the binding of a Wnt-protein ligand to a Frizzled family receptor, with biological signals passing to the Disheveled protein inside the cell. The canonical Wnt pathway leads to regulation of gene transcription, while noncanonical pathways regulate the cytoskeleton and intracellular calcium, for example. Canonical Wnt signaling pathways involve β-catenin. By contrast, non-canonical Wnt signaling operates independent of β-catenin.

“Subject,” as used herein refers to any vertebrate animal, including mammals and non-mammals. A subject can include primates, including humans, and non-primate mammals, such as rodents, domestic animals or game animals. Non-primate mammals can include mouse, rat, hamster, rabbit, dog, fox, wolf, cat, horse, cow, pig, sheep, goat, camel, deer, buffalo, bison, etc. Non-mammals can include bird (e.g., chicken, ostrich, emu, pigeon), reptile (e.g., snake, lizard, turtle), amphibian (e.g., frog, salamander), fish (e.g., salmon, cod, pufferfish, tuna), etc. The terms, “individual,” “patient,” and “subject” are used interchangeably herein.

As used herein, “treat” and “treatment” includes curing, improving, ameliorating, reducing the severity of, preventing, slowing the progression of, and/or delaying the appearance of a disease, condition and/or symptoms thereof.

A treatment can be considered “effective,” or “therapeutically effective” as used herein, if one or more of the signs or symptoms of a condition described herein are altered in a beneficial manner, other clinically accepted symptoms are improved, or even ameliorated, or a desired response is induced e.g., by at least 2%, 3%, 4%, 5%, 10%, or more, following treatment according to the methods described herein. Efficacy can be assessed, for example, by measuring a marker, indicator, symptom, and/or the incidence of a condition treated according to the methods described herein or any other measurable parameter appropriate, e.g. electrical activity in the heart. Efficacy can also be measured by a failure of an individual to worsen as assessed by hospitalization, or need for medical interventions (e.g., progression of the disease is halted). Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. heart activity). One skilled in the art can monitor efficacy of administration and/or treatment by measuring any one of such parameters, or any combination of parameters.

The term “effective amount” or “therapeutically effective amount” as used herein refers to the amount of a composition or an agent needed to alleviate at least one or more symptom of the disease or disorder, and relates to a sufficient amount of therapeutic composition to provide the desired effect. “Effective amount” or “therapeutically effective amount” can refer to an amount of a composition or therapeutic agent that is sufficient to provide a particular reparative and/or regenerative effect when administered to a typical subject. A therapeutically effective amount as used herein, in various contexts, can include an amount sufficient to delay the development of a symptom of the disease, alter the course of a symptom disease (for example but not limited to, slowing the progression of a symptom of the disease), or reverse a symptom of the disease. The therapeutically effective amount may be administered in one or more doses of the therapeutic agent. The therapeutically effective amount may be administered in a single administration, or over a period of time in a plurality of doses.

“Administering” as used herein can include any suitable routes of administering a therapeutic agent or composition as disclosed herein. Suitable routes of administration include, without limitation, oral, parenteral, intravenous, intramuscular, subcutaneous, transdermal, airway (aerosol), pulmonary, cutaneous, injection or topical administration. Administration can be local or systemic.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein the term “nucleic acid” refers to multiple nucleotides (i.e. molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). The term also includes polynucleosides (i.e. a polynucleotide minus the phosphate) and any other organic base containing polymer. Purines and pyrimidines include but are not limited to adenine, cytosine, guanine, thymidine, inosine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Any suitable modifications are contemplated. Thus, the term nucleic acid also encompasses nucleic acids with substitutions or modifications, such as in the bases and/or sugars.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-91 1910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

### Methods

Provided herein are methods of treating a condition, e.g., a condition requiring tissue repair and/or regeneration, by administering exosome-derived PIWI-interacting RNA (piRNA) to a subject in need thereof. A variety of conditions can be treated by the present methods. Conditions include, without limitation, ischemic injury (e.g., ischemic injury to muscle), damage to tissue (e.g., muscle tissue) after ischemia/reperfusion, and tissue fibrosis. In some embodiments, the condition is one that causes tissue fibrosis, e.g., if left untreated.

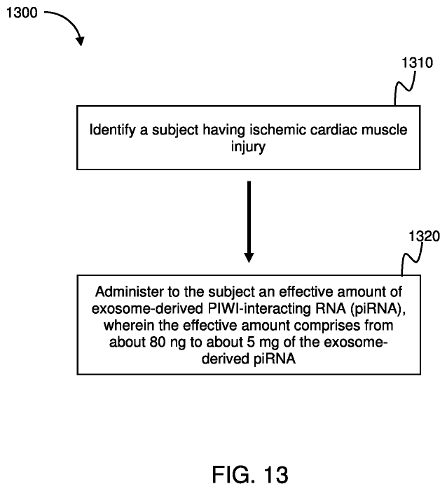

Provided herein in certain embodiments are cell-free and/or exosome-free methods of treating ischemic cardiac muscle injury. With reference to FIG. 13, a non-limiting example of an embodiment of a method of treating ischemic cardiac muscle injury is described. The method 1300 includes identifying 1310 a subject having or in need of treating an ischemic cardiac muscle injury, and administering 1320 to the subject an effective (or therapeutically effective) amount of exosome-derived PIWI-interacting RNA (piRNA), e.g., CDC (cardiosphere-derived cells)-derived exosomal piRNA. The effective (or therapeutically effective) amount of piRNA administered can be in a range of about 80 ng to about 5 mg, e.g., a range of about 80 ng to about 500 μg.

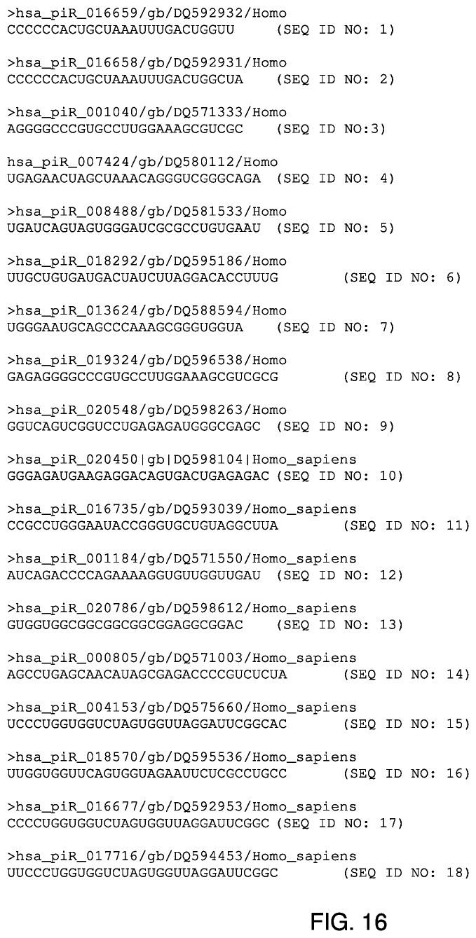

Also provided is a method of treating ischemic cardiac muscle injury that includes identifying a subject having or in need of treating an ischemic cardiac muscle injury, and administering to the subject a therapeutically effective amount of an RNA (e.g., piRNA, such as hsa_piR_016659), to thereby treat the ischemic cardiac muscle injury. In some embodiments, the piRNA includes a nucleotide sequence of hsa_piR_016659 (SEQ ID NO: 1), e.g., as shown in FIG. 16, or a variant or derivative thereof. In some embodiments, the effective (or therapeutically effective) amount of piRNA administered is in a range of about 80 ng to about 5 mg, e.g., a range of about 80 ng to about 500 μg.

A variety of ischemic cardiac muscle injury can be treated by the present methods. In some embodiments, ischemic cardiac muscle injury comprises damage to cardiac muscle due to ischemia. In some embodiments, ischemic cardiac muscle injury includes ischemic/reperfusion injury, e.g., damage to cardiac muscle from reperfusion following ischemia. In some embodiments, ischemic cardiac muscle injury includes cardiac muscle fibrosis. In some embodiments, the subject has suffered a myocardial infarction.

The piRNA can be administered at any suitable time. In some embodiments, the piRNA is administered about 10 minutes, about 20 minutes, about 30 minutes, about 45 minutes, about 60 minutes, about 90 minutes, about 2 hours, about 3 hours, about 4 hours, about 6 hours, about 12 hours, about 24 hour or more, or a time interval in a range defined by any two of the preceding values, after the subject suffers the ischemic cardiac muscle injury. In several embodiments, piRNA can be administered prophylactically, such as to a subject exhibiting preliminary symptoms or at extremely high risk for an ischemic event.

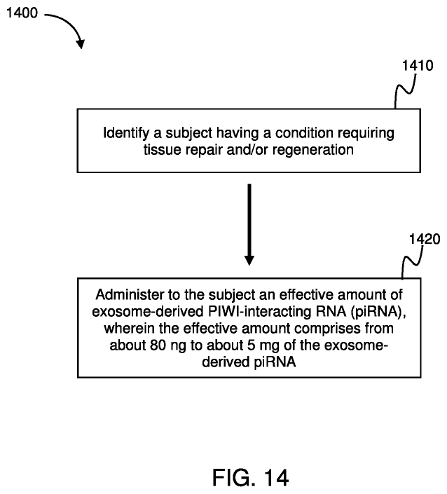

Provided herein are exosome-free methods of treating a condition requiring tissue repair and/or regeneration. With reference to FIG. 14, a non-limiting example of an embodiment of a method of treating a condition requiring tissue repair and/or regeneration is described. The method 1400 includes identifying 1410 a subject having condition requiring tissue repair and/or regeneration, and administering 1420 to the subject an effective (or therapeutically effective) amount of exosome-derived PIWI-interacting RNA (piRNA). The effective (or therapeutically effective) amount of piRNA administered can be in a range of about 80 ng to about 5 mg, e.g., a range of about 80 ng to about 500 μg.

A variety of condition requiring tissue repair and/or regeneration can be treated by the present methods. In some embodiments, the condition includes injury or damage to muscle or lung tissue. In some embodiments, the condition includes injury or damage to skeletal muscle or cardiac muscle. In some embodiments, the condition includes or is a condition that causes tissue fibrosis, e.g., cardiac muscle fibrosis or pulmonary fibrosis. In some embodiments, the condition includes ischemic cardiac muscle injury, e.g., as described herein. In some embodiments, the condition includes pulmonary fibrosis, e.g., idiopathic pulmonary fibrosis.

Any suitable amount of piRNA can be administered. In some embodiments, the effective (or therapeutically effective) amount of piRNA is about 80 ng, about 100 ng, about 120 ng, about 140 ng, about 160 ng, about 180 ng, about 200 ng, about 250 ng, about 300 ng, about 350 ng, about 400 ng, about 500 ng, about 600 ng, about 700 ng, about 800 ng, about 900 ng, about 1 μg, about 2 μg, about 5 μg, about 10 μg, about 20 μg, about 50 μg, about 100 μg, about 200 μg, about 500 μg, about 1 mg, about 2 mg, about 5 mg, or more, or an amount in a range defined by any two of the preceding values. In certain embodiments, the piRNA is administered on a per kilogram basis, for example, about 100 ng/kg to about 10 mg/kg of body weight, e.g., about 1 μg/kg to about 1 mg/kg, including about 1 μg/kg to about 100 μg/kg. In additional embodiments, exosomes are delivered in an amount based on the mass of the target tissue (e.g., heart or lung), for example about 1 μg/kg to about 100 mg/kg of target tissue, e.g., about 10 μg/kg to about 100 mg/kg, about 100 μg/kg to about 10 mg/kg, including about 1 mg/kg to about 10 mg/kg of the target tissue. In some embodiments, the effective (or therapeutically effective) amount of exosome-derived piRNA is an amount having a therapeutic effect equivalent to a therapeutic effect of administering about 10⁹, about 2×10⁹, about 5×10⁹, about 10¹⁰, about 2×10¹⁰, about 5×10¹⁰, about 10¹¹, about 2×10¹¹, about 5×10¹², about 10¹² or more, or a number in a range defined by any two of the preceding values, of immortalized CDC-derived exosomes. In some embodiments, the piRNA is hsa_piR_016659.

The exosome-derived piRNA can be administered via any suitable route of administration. In some embodiments, the piRNA is administered systemically. In some embodiments, the piRNA is administered locally. Local administration, depending on the tissue to be treated, may in some embodiments be achieved by direct administration to a tissue (e.g., direct injection, such as intramyocardial injection). Local administration may also be achieved by, for example, lavage of a particular tissue (e.g., intra-tracheal lavage). In some embodiments, the exosomes and/or piRNA are nebulized or inhaled. In some embodiments, the piRNA is administered parenterally. In some embodiments, the exosome-derived piRNA is administered intravenously, intra-arterially, intramuscularly, intracardially, or intratracheally.

In general, piRNA of the present methods are derived from, and/or are isolated from, exosomes, e.g., exosomes derived from therapeutic cells, as described herein. In some embodiments, methods of the present disclosure are exosome-free methods. In some embodiments, no substantial amount of exosome is administered with the piRNA to the subject. In some embodiments, the piRNA is administered essentially free of exosomes. In some embodiments, substantially all of the administered piRNA is not associated with exosomes. In some embodiments, about 5% or less, about 4% or less, about 3% or less, about 2% or less, about 1% or less, about 0.5% or less, about 0.2% or less, about 0.1% or less, about 0.05% or less, about 0.02% or less, about 0.01% or less, about 0.001% or less, about 0.0001% or less, about 0.00001% or less, or a percentage in a range defined by any two of the preceding values, of the administered piRNA is associated with exosomes. In some embodiments, a composition comprising the piRNA and administered to the subject is substantially free of exosomes.

In some embodiments, the method is cell-free method of treating a condition requiring tissue repair and/or regeneration. In some embodiments, the cell-free method may include administering the effective (or therapeutically effective) amount of exosome-derived piRNA via administration of exosomes, extracellular vesicles, and/or liposomes (e.g., synthetic liposomes) containing the exosome-derived piRNA. In some embodiments, the exosomes, extracellular vesicles, and/or liposomes are enriched for the exosome-derived piRNA. In some embodiments, the exosome-derived piRNA is enriched compared to the amount of exosome-derived piRNA in exosomes derived from therapeutic cells (e.g., immortalized CDCs, engineered fibroblasts (ASTECs)).

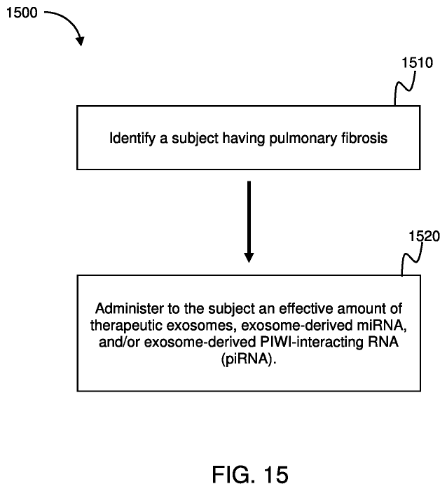

Also provided herein are cell-free methods of treating pulmonary fibrosis. With reference to FIG. 15, a non-limiting example of an embodiment of a method of treating a pulmonary fibrosis is described. The method 1500 includes identifying 1510 a subject having pulmonary fibrosis, and administering 1520 to the subject an effective (or therapeutically effective) amount of therapeutic exosomes and/or exosome-derived PIWI-interacting RNA (piRNA). The effective (or therapeutically effective) amount of piRNA administered can be in a range of about 80 ng to about 5 mg, e.g., a range of about 80 ng to about 500 μg. The effective (or therapeutically effective) amount of therapeutic exosomes administered can be about 10⁶ to about 10¹² particles.

Any suitable amount of the therapeutic exosomes can be administered to the subject. In some embodiments, the present methods include administering from about 10⁶ to about 2×10⁶ particles, about 2×10⁶ to about 5×10⁶ particles, about 10⁷ to about 2×10⁷ particles, about 2×10⁷ to about 5×10⁷ particles, about 5×10⁷ to about 10⁸ particles, about 10⁸ to about 2×10⁸ particles, about 2×10⁸ to about 5×10⁸ particles, about 5×10⁸ to about 10⁹ particles, about 10⁹ to about 2×10⁹ particles, about 2×10⁹ to about 5×10⁹ particles, about 5×10⁹ to about 10¹⁰ particles, about 10¹⁰ to about 2×10¹⁰ particles, about 2×10¹⁰ to about 5×10¹⁰ particles, about 5×10¹⁰ to about 10¹¹ particles, about 10¹¹ to about 2×10¹¹ particles, about 2×10¹¹ to about 5×10¹¹ particles, or about 5×10¹¹ to about 10¹² particles of the therapeutic exosomes.

In certain embodiments, the exosome dose is administered on a per kilogram basis, for example, about 1.0×10⁵ exosomes/kg to about 1.0×10⁹ exosomes/kg. In additional embodiments, exosomes are delivered in an amount based on the mass of the target tissue, for example about 1.0×10⁵ exosomes/gram of target tissue to about 1.0×10⁹ exosomes/gram of target tissue. In several embodiments, exosomes are administered based on a ratio of the number of exosomes the number of cells in a particular target tissue, for example exosome:target cell ratio ranging from about 10⁹:1 to about 1:1, including about 10⁸:1, about 10⁷:1, about 10⁶:1, about 10⁵:1, about 10⁴:1, about 10³:1, about 10²:1, about 10:1, and ratios in between these ratios. In additional embodiments, exosomes are administered in an amount about 10-fold to an amount of about 1,000,000-fold greater than the number of cells in the target tissue, including about 50-fold, about 100-fold, about 500-fold, about 1000-fold, about 10,000-fold, about 100,000-fold, about 500,000-fold, about 750,000-fold, and amounts in between these amounts. If the exosomes are to be administered in conjunction with the concurrent therapy (e.g., cells that can still shed exosomes, pharmaceutical therapy, nucleic acid therapy, and the like) the dose of exosomes administered can be adjusted accordingly (e.g., increased or decreased as needed to achieve the desired therapeutic effect).

The therapeutic exosomes and/or exosome-derived piRNA can be administered via any suitable route of administration. In some embodiments, the exosomes and/or piRNA are administered systemically. In some embodiments, the exosomes and/or piRNA are administered locally. In some embodiments, the exosomes and/or piRNA are administered parenterally. In some embodiments, the exosomes and/or piRNA are administered intratracheally, e.g., by intratracheal lavage. In some embodiments, the exosomes and/or piRNA are nebulized or inhaled.

In several embodiments, the piRNA and/or exosomes are delivered in a single, bolus dose. In some embodiments, however, multiple doses of piRNA and/or exosomes may be delivered. In certain embodiments, piRNA and/or exosomes can be infused (or otherwise delivered) at a specified rate over time. In several embodiments, when piRNA and/or exosomes are administered within a relatively short time frame after an adverse event (e.g., an injury or damaging event, or adverse physiological event such as an MI), their administration prevents the generation or progression of damage to a target tissue. For example, if piRNA and/or exosomes are administered within about 20 to about 30 minutes, within about 30 to about 40 minutes, within about 40 to about 50 minutes, within about 50 to about 60 minutes post-adverse event, the damage or adverse impact on a tissue is reduced (as compared to tissues that were not treated at such early time points). In some embodiments, the administration is as soon as possible after an adverse event. In some embodiments the administration is as soon as practicable after an adverse event (e.g., once a subject has been stabilized in other respects). In several embodiments, administration is within about 1 to about 2 hours, within about 2 to about 3 hours, within about 3 to about 4 hours, within about 4 to about 5 hours, within about 5 to about 6 hours, within about 6 to about 8 hours, within about 8 to about 10 hours, within about 10 to about 12 hours, and overlapping ranges thereof. Administration at time points that occur longer after an adverse event are effective at preventing damage to tissue, in certain additional embodiments. As mentioned above, in several embodiments piRNA and/or exosomes can be administered prophylactically.

In several embodiments, the exosomes are specifically targeted to the damaged or diseased tissues. In some such embodiments, the exosomes are modified (e.g., genetically or otherwise) to direct them to a specific target site. For example, modification may, in some embodiments, comprise inducing expression of a specific cell-surface marker on the exosome, which results in specific interaction with a receptor on a desired target tissue. In one embodiment, the native contents of the exosome are removed and replaced with desired exogenous proteins or nucleic acids. In one embodiment, the native contents of exosomes are supplemented with desired exogenous proteins or nucleic acids. In some embodiments, however, targeting of the exosomes is not performed. In several embodiments, exosomes are modified to express specific nucleic acids or proteins, which can be used, among other things, for targeting, purification, tracking, etc. In several embodiments, however, modification of the exosomes is not performed. In some embodiments, the exosomes do not comprise chimeric molecules.

In general, the therapeutic exosomes of the present methods are derived from, and/or are isolated from, therapeutic cells, as described herein. In some embodiments, methods of the present disclosure are cell-free methods. In some embodiments, no substantial amount of cells is administered with the therapeutic exosomes and/or piRNA to the subject. In some embodiments, the therapeutic exosomes and/or the piRNA is administered essentially free of cells. In some embodiments, substantially all of the administered therapeutic exosomes and/or piRNA is not associated with cells. In some embodiments, about 5% or less, about 4% or less, about 3% or less, about 2% or less, about 1% or less, about 0.5% or less, about 0.2% or less, about 0.1% or less, about 0.05% or less, about 0.02% or less, about 0.01% or less, about 0.001% or less, about 0.0001% or less, about 0.00001% or less, or a percentage in a range defined by any two of the preceding values, of the administered therapeutic exosomes and/or piRNA is associated with cells. In some embodiments, a composition comprising the therapeutic exosomes and/or piRNA, and administered to the subject is substantially free of cells. In some embodiments, the exosome-derived piRNA is synthetic RNA generated using any suitable option for nucleic acid synthesis. In some embodiments, the exosome-derived piRNA is chemically synthesized. In some embodiments, synthetic exosome-derived piRNA includes natural and/or non-natural nucleotides. In some embodiments, synthetic exosome-derived piRNA includes nucleotide analogues and derivatives. In some embodiments, the exosome-derived piRNA is produced recombinantly.

A variety of piRNA, e.g., exosome-derived piRNA, can be used in the present methods, as described herein. In some embodiments, the exosome-derived piRNA is fibroblast-derived exosomal piRNA, e.g., piRNA derived from therapeutic exosomes from engineered fibroblasts or ASTEX, as described herein. In some embodiments, the method includes treating pulmonary fibrosis by administering fibroblast-derived exosomal piRNA. In some embodiments, the piRNA includes one or more of piR-20450, piR-20548, piR-16735, piR-01184, piR-20786, piR-00805, piR-04153, piR-18570, piR-16677, and piR-17716, e.g., as shown in FIG. 16.

In some embodiments, the piRNA, e.g., exosome-derived piRNA, is CDC-derived exosomal piRNA, e.g., piRNA derived from, or enriched in, therapeutic exosomes from immortalized CDCs (imCDCs), as described herein. In some embodiments, the piRNA includes one or more of hsa_piR_016659, hsa_piR_016658, hsa_piR_001040, hsa_piR_007424, hsa_piR_008488, hsa_piR_018292, hsa_piR_013624, hsa_piR_019324, and hsa_piR_020548, e.g., as shown in FIG. 16. In some embodiments, the piRNA includes a sequence at least 85%, at least 90%, at least 95%, at least 96%, at least 97%, at least 98%, or more identical to one or more of hsa_piR_016659, hsa_piR_016658, hsa_piR_001040, hsa_piR_007424, hsa_piR_008488, hsa_piR_018292, hsa_piR_013624, hsa_piR_019324, and hsa_piR_020548, e.g., as shown in FIG. 16. In some embodiments, the piRNA includes a sequence that differs by no more than 1, 2, 3, 4, or 5 nucleotides from one or more of hsa_piR_016659, hsa_piR_016658, hsa_piR_001040, hsa_piR_007424, hsa_piR_008488, hsa_piR_018292, hsa_piR_013624, hsa_piR_019324, and hsa_piR_020548. In some embodiments, the piRNA is hsa_piR_016659, e.g., as shown in FIG. 16. In some embodiments, the piRNA includes a sequence at least 85%, at least 90%, at least 95%, at least 96%, at least 97%, at least 98%, or more identical to hsa_piR_016659, e.g., as shown in FIG. 16. In some embodiments, the piRNA includes a sequence that differs by no more than 1, 2, 3, 4, or 5 nucleotides from hsa_piR_016659, e.g., as shown in FIG. 16.

In some embodiments, the method includes isolating exosome-derived piRNA from therapeutic exosomes. In some embodiments, the method includes isolating the therapeutic exosomes from a population of therapeutic cells, e.g., engineered fibroblasts or immortalized CDCs (imCDCs). In some embodiments, the method includes generating the population of therapeutic cells from non-therapeutic cells, e.g., engineered fibroblasts from normal human dermal fibroblasts, or enhanced potency imCDCs from low potency imCDCs. Any suitable option for generating a population of therapeutic cells from non-therapeutic cells can be used. In some embodiments, a population of therapeutic cells is produced by activating Wnt/βcatenin signaling in non-therapeutic cells, e.g., low therapeutic potency imCDCs, to thereby generate enhanced therapeutic potency imCDCs. As used herein, “immortalized CDC” and “imCDC” may be used interchangeably to refer to enhanced therapeutic potency imCDC, unless indicated otherwise. In some embodiments, a population of therapeutic cells is produced by activating Wnt/βcatenin signaling and overexpressing gata4 in fibroblasts, e.g., normal human dermal fibroblasts, to thereby generate engineered fibroblasts. As used herein, “ASTEX” refer to extracellular vesicles/exosomes derived from engineered fibroblasts. Suitable options for generating a population of therapeutic cells from non-therapeutic cells are described, e.g., in International Application No. PCT/US20/31808 and Ibrahim et al., Nat Biomed Eng. 2019 September; 3(9):695-705, which disclosures are incorporated herein by reference in their entirety.

In several embodiments, exosomes (e.g., exosomes engineered for high potency), can be manipulated, for example through gene editing using, for example CRISPR-Cas, zinc finger nucleases, and/or TALENs, to reduce their potential immunogenicity. Advantageously, the exosomes can be derived, depending on the embodiment, from cells obtained from a source that is allogeneic, autologous, xenogeneic, or syngeneic with respect to the eventual recipient of the exosomes. Moreover, master banks of exosomes that have been characterized for their expression of certain piRNAs, miRNAs and/or proteins can be generated and stored long-term for subsequent use in defined subjects on an “off-the-shelf” basis. However, in several embodiments, exosomes are isolated and then used without long-term or short-term storage (e.g., they are used as soon as practicable after their generation).

In some embodiments, exosomes are harvested as described herein and subjected to methods to liberate and collect their nucleic acid contents, e.g., piRNA. In several embodiments, nucleic acids are isolated using chaotropic disruption of the exosomes and subsequent isolation of nucleic acids. Other established methods for nucleic acid isolation may also be used in addition to, or in place of chaotropic disruption. Nucleic acids that are isolated may include, but are not limited to DNA, DNA fragments, and DNA plasmids, total RNA, mRNA, tRNA, snRNA, saRNA, miRNA, piRNA, rRNA, regulating RNA, non-coding and coding RNA, and the like. In several embodiments in which RNA is isolated, the RNA can be used as a template in an RT-PCR-based (or other amplification) method to generate large copy numbers (in DNA form) of the RNA of interest. In such instances, should a particular RNA or fragment be of particular interest, the exosomal isolation and preparation of the RNA can optionally be supplemented by the in vitro synthesis and co-administration of that desired sequence.

In several embodiments, the piRNA and/or exosomes are administered in combination with one or more additional agents. For example, in several embodiments, the piRNA and/or exosomes are administered in combination with one or more proteins or nucleic acids derived from the exosome (e.g., to supplement the exosomal contents). In several embodiments, the cells from which the piRNA and/or exosomes are isolated are administered in conjunction with the exosomes.

In several embodiments, the piRNA and/or exosomes are delivered in conjunction with a more traditional therapy, e.g., surgical therapy or pharmaceutical therapy. In several embodiments such combinations of approaches result in synergistic improvements in the viability and/or function of the target tissue. In some embodiments, exosomes may be delivered in conjunction with a gene therapy vector (or vectors), nucleic acids (e.g., those used as siRNA or to accomplish RNA interference), and/or combinations of exosomes derived from other cell types.

Thus, in some embodiments, delivery of piRNA and/or exosomes (alone or in combination with an adjunct agent such as nucleic acid) provide certain effects (e.g., paracrine effects) that serve to promote repair of tissue, improvement in function, increased viability, or combinations thereof. In some embodiments, the piRNA content of delivered exosomes is responsible for at least a portion of the repair or regeneration of a target tissue. In several embodiments, miRNA delivery by exosomes is responsible, in whole or in part, for repair and/or regeneration of damaged tissue. As discussed above, miRNA delivery may operate to repress translation of certain messenger RNA (for example, those involved in programmed cell death), or may result in messenger RNA cleavage. In either case, and in some embodiments, in combination, these effects alter the cell signaling pathways in the target tissue and, as demonstrated by the data disclosed herein, can result in improved cell viability, increased cellular replication, beneficial anatomical effects, and/or improved cellular function, each of which in turn contributes to repair, regeneration, and/or functional improvement of a damaged or diseased tissue as a whole.

The beneficial effects of the piRNA and/or exosomes (or their contents) need not only be on directly damaged or injured cells. In some embodiments, for example, the cells of the damaged tissue that are influenced by the disclosed methods are healthy cells. However, in several embodiments, the cells of the damaged tissue that are influenced by the disclosed methods are damaged cells.

In several embodiments, regeneration comprises improving the function of the tissue. For example, in certain embodiments in which cardiac tissue is damaged, functional improvement may comprise increased cardiac output, contractility, ventricular function and/or reduction in arrhythmia (among other functional improvements). For other tissues, improved function may be realized as well, such as enhanced cognition in response to treatment of neural damage, improved blood-oxygen transfer in response to treatment of lung damage, improved immune function in response to treatment of damaged immunological-related tissues.

In several embodiments, the regenerative piRNA and/or exosomes are mammalian in origin. In several embodiments, the regenerative piRNA and/or exosomes are human in origin. In some embodiments, the piRNA and/or exosomes are derived from non-embryonic human regenerative cells and/or exosomes. In several embodiments, the regenerative exosomes are autologous to the individual while in several other embodiments the regenerative exosomes are allogeneic to the individual. Xenogeneic or syngeneic exosomes are used in certain other embodiments.

Also provided herein are methods of regulating tissue repair, comprising contacting a population of transdifferentiating fibroblasts with an effective (or therapeutically effective) amount of exosomes, exosome-derived miRNA, and/or exosome-derived PIWI-interacting RNA (piRNA), to thereby suppress transdifferentiation of the fibroblasts into myofibroblasts, wherein the exosomes are derived from engineered fibroblasts. In some embodiments, the transdifferentiation is TGFβ-mediated transdifferentiation, e.g., transdifferentiation induced by TGFβ signaling. In some embodiments, TGFβ signaling is activated by tissue injury or damage. In some embodiments, the fibroblasts are lung fibroblasts.

In some embodiments, the contacting comprises administering the exosomes, exosome-derived miRNA, and/or exosome-derived piRNA to a subject. In some embodiments, the subject has pulmonary fibrosis, e.g., idiopathic pulmonary fibrosis. In some embodiments, the contacting comprises administering the exosomes, exosome-derived miRNA, and/or exosome-derived piRNA to a subject intratracheally, e.g., by intratracheal lavage, inhalation or nebulization. Any suitable amount of exosomes can be contacted with transdifferentiating fibroblasts or administered to the subject, as described herein. In some embodiments, the effective (or therapeutically effective) amount of exosomes comprises about 10⁶ to about 10¹² particles.

### Therapeutic Cells, Exosomes Derived Therefrom, and PIWI-Interacting RNA

PIWI-interacting RNA (piRNA) for use in the present methods are generally derived from extracellular vesicles (EVs), e.g., exosomes derived from therapeutic cells. Suitable EVs or exosomes from which piRNA can be derived include exosomes derived from CDCs, e.g., immortalized CDCs, and exosomes derived from engineered fibroblasts, e.g., ASTEX.

Certain types of nucleic acids are associated with membrane-bound particles. Such membrane-bound particles are shed from most cell types and consist of fragments of plasma membrane and contain DNA, RNA, mRNA, microRNA, piRNA and proteins. These particles often mirror the composition of the cell from which they are shed. Exosomes are one type of such membrane bound particles and typically range in diameter from about 15 nm to about 95 nm in diameter, including about 15 nm to about 20 nm, 20 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 95 nm, and overlapping ranges thereof In several embodiments, exosomes are larger (e.g., those ranging from about 140 to about 210 run, including about 140 nm to about 150 nm, 150 nm to about 160 run, 160 nm to about 170 run, 170 nm to about 180 nm, 180 nm to about 190 run, 190 nm to about 200 run, 200 nm to about 210 nm, and overlapping ranges thereof). In some embodiments, the exosomes that are generated from the original cellular body are 100, 200, 300, 400, 500, 600, 700, 800, 900, 1000, 2000, 5000, 10,000 times smaller in at least one dimension (e.g., diameter) than the original cellular body.

Alternative nomenclature is also often used to refer to exosomes. Thus, as used herein the term “exosome” shall be given its ordinary meaning and may also include terms including microvesicles, epididimosomes, argosomes, exosome-like vesicles, microparticles, promininosomes, prostasomes, dexosomes, texosomes, dex, tex, archeosomes and oncosomes. Unless otherwise indicated herein, each of the foregoing terms shall also be understood to include engineered high-potency varieties of each type of exosome. Exosomes are secreted by a wide range of mammalian cells and are secreted under both normal and pathological conditions. Exosomes, in some embodiments, function as intracellular messengers by virtue of carrying mRNA, miRNA, piRNA or other contents from a first cell to another cell (or plurality of cells).

Exosomes, in several embodiments, are isolated from cellular preparations by methods comprising one or more of filtration, centrifugation, antigen-based capture and the like. For example, in several embodiments, a population of cells grown in culture are collected and pooled. In several embodiments, monolayers of cells are used, in which case the cells are optionally treated in advance of pooling to improve cellular yield (e.g., dishes are scraped and/or enzymatically treated with an enzyme such as trypsin to liberate cells). In some embodiments, cells are cultured under serum starvation for about 10 days or more, about 12 days or more, or about 15 days or more, and exosomes are collected from the conditioned medium. In some embodiments, cells grown in culture under standard cell culture conditions are exposed to serum-free medium under hypoxic condition overnight, and conditioned media containing exosomes are collected. In some embodiments, the hypoxic condition includes about 15%, about 12%, about 10%, about 9%, about 8%, about 7%, about 6%, about 5%, about 4%, about 3%, about 2%, about 1%, O₂ or less, or a percentage of O₂ in a range defined by any two of the preceding values. In some embodiments, the hypoxic condition includes 2% O₂/5% CO₂ at 37° C. In some embodiments, the cells exposed to hypoxic condition recover in complete serum under standard oxygen at 37° C. for about 24, about 36, about 48, about 60, about 72 hours or more, or a time interval in a range defined by any two of the preceding values, and are then re-exposed to hypoxic condition to generate condition media. The standard oxygen includes about 15%, about 16%, about 17%, about 18%, about 19%, about 20%, about 21%, about 22%, about 23% about 24%, about 25%, or more O₂, a percentage of O₂ in a range between any two of the preceding values. In some embodiments, cells are cycled between hypoxic and standard oxygen media for 1, 2, 3, 4, 5, 6 or more times. In several embodiments, cells grown in suspension are used. The pooled population is then subject to one or more rounds of centrifugation (in several embodiments ultracentrifugation and/or density centrifugation is employed) in order to separate the exosome fraction from the remainder of the cellular contents and debris from the population of cells. In some embodiments, centrifugation need not be performed to harvest exosomes. In several embodiments, pre-treatment of the cells is used to improve the efficiency of exosome capture. For example, in several embodiments, agents that increase the rate of exosome secretion from cells are used to improve the overall yield of exosomes. In some embodiments, augmentation of exosome secretion is not performed. In some embodiments, size exclusion filtration is used in conjunction with, or in place of centrifugation, in order to collect a particular size (e.g., diameter) of exosome. In some embodiments, exosomes are purified using centrifugal ultrafiltration with a 1000 KDa molecular weight cutoff filter. In several embodiments, filtration need not be used. In still additional embodiments, exosomes (or subpopulations of exosomes are captured by selective identification of unique markers on or in the exosomes (e.g., transmembrane proteins)). In such embodiments, the unique markers can be used to selectively enrich a particular exosome population. In some embodiments, enrichment, selection, or filtration based on a particular marker or characteristic of exosomes is not performed.

In addition, methods are provided for facilitating the generation of exosomes, and in particular exosomes engineered for high potency. In several such embodiments, a hydrolase is used to facilitate the liberation (e.g., secretion) of exosomes from cells. In certain embodiments, hydrolases that cleave one or more of ester bonds, sugars (e.g., DNA), ether bonds, peptide bonds, carbon-nitrogen bonds, acid anhyrides, carbon-carbon bonds, halide bonds, phosphorous-nitrogen bonds, sulpher-nitrogen bonds, carbon-phosphorous bonds, sulfur-sulfur bonds, and/or carbon-sulfur bonds are used. In some embodiments, the hydrolases are DNAses (e.g., cleave sugars). Certain embodiments employ specific hydrolases, such as for example, one or more of lysosomal acid sphingomyelinase, secreted zinc-dependent acid sphingomyelinase, neutral sphingomyelinase, and alkaline sphingomyelinase.

In several embodiments, exosomes are administered to a subject in order to initiate the repair or regeneration of cells or tissue. In several embodiments, the exosomes are derived from a stem cell. In several embodiments, the stem cells are non-embryonic stem cells. In some embodiments, the non-embryonic stem cells are adult stem cells. However, in certain embodiments, embryonic stem cells are optionally used as a source for exosomes. In some embodiments, somatic cells (by way of non-limiting example, fibroblasts) are used as a source for exosomes. In still additional embodiments, germ cells are used as a source for exosomes.

In some embodiments, cells with high therapeutic potency are generated, as described herein. In some embodiments, cells are engineered to produce exosomes of high therapeutic potency. Any cell type can be used to generate cells with high therapeutic potency and/or that produce exosomes of high therapeutic potency. For example, cardioshpere derived cells (CDCs) or fibroblast cells can be used.

In several embodiments employing stem cells as an exosome source, the nucleic acid and/or protein content of exosomes from stem cells are particularly suited to effect the repair or regeneration of damaged or diseased cells. In several embodiments, exosomes are isolated from stem cells derived from the tissue to be treated. For example, in some embodiments where cardiac tissue is to be repaired, exosomes are derived from cardiac stem cells. Cardiac stem cells are obtained, in several embodiments, from various regions of the heart, including but not limited to the atria, septum, ventricles, auricola, and combinations thereof (e.g., a partial or whole heart may be used to obtain cardiac stem cells in some embodiments). In several embodiments, exosomes are derived from cells (or groups of cells) that comprise cardiac stem cells or can be manipulated in culture to give rise to cardiac stem cells (e.g., cardiospheres and/or cardiosphere derived cells (CDCs)). Further information regarding the isolation of cardiospheres can be found in U.S. Pat. No. 8,268,619, issued on Sep. 18, 2012, which is incorporated in its entirety by reference herein. In several embodiments, the cardiac stem cells are cardiosphere-derived cells (CDCs). Further information regarding methods for the isolation of CDCs can be found in U.S. patent application Ser. No. 11/666,685, filed on Apr. 21, 2008, and Ser. No. 13/412,051, filed on Mar. 5, 2012, both of which are incorporated in their entirety by reference herein. Other varieties of stem cells may also be used, depending on the embodiment, including but not limited to bone marrow stem cells, adipose tissue derived stem cells, mesenchymal stem cells, induced pluripotent stem cells, hematopoietic stem cells, and neuronal stem cells.

In several embodiments, the exosomes induce altered gene expression by repressing translation and/or cleaving mRNA, for example. In some embodiments, the alteration of gene expression results in inhibition of undesired proteins or other molecules, such as those that are involved in cell death pathways, or induce further damage to surrounding cells (e.g., free radicals). In several embodiments, the alteration of gene expression results directly or indirectly in the creation of desired proteins or molecules (e.g., those that have a beneficial effect). The proteins or molecules themselves need not be desirable per se (e.g., the protein or molecule may have an overall beneficial effect in the context of the damage to the tissue, but in other contexts would not yield beneficial effects). In some embodiments, the alteration in gene expression causes repression of an undesired protein, molecule or pathway (e.g., inhibition of a deleterious pathway). In several embodiments, the alteration of gene expression reduces the expression of one or more inflammatory agents and/or the sensitivity to such agents. Advantageously, the administration of exosomes, miRNAs, or piRNAs in several embodiments, results in downregulation of certain inflammatory molecules and/or molecules involved in inflammatory pathways. As such, in several embodiments, cells that are contacted with the exosomes, miRNAs, or piRNAs enjoy enhanced viability, even in the event of post-injury inflammation or inflammation due to disease.

In several embodiments, the exosomes fuse with one or more recipient cells of the damaged tissue. In several embodiments, the exosomes release the microRNA and/or piRNA into one or more recipient cells of the damaged tissue, thereby altering at least one pathway in the one or more cells of the damaged tissue. In some embodiments, the exosomes exerts their influence on cells of the damaged tissue by altering the environment surrounding the cells of the damaged tissue. In some embodiments, signals generated by or as a result of the content or characteristics of the exosomes, lead to increases or decreases in certain cellular pathways. For example, the exosomes (or their contents/characteristics) can alter the cellular milieu by changing the protein and/or lipid profile, which can, in turn, lead to alterations in cellular behavior in this environment. Additionally, in several embodiments, the miRNA and/or piRNA of an exosome can alter gene expression in a recipient cell, which alters the pathway in which that gene was involved, which can then further alter the cellular environment. In several embodiments, the influence of the exosomes directly or indirectly stimulates angiogenesis. In several embodiments, the influence of the exosomes directly or indirectly affects cellular replication. In several embodiments, the influence of the exosomes directly or indirectly inhibits cellular apoptosis. Likewise, in several embodiments, piRNA derived from exosomes induces these and/or other effects.

### Therapeutic Compositions

In several embodiments, there are provided compositions, e.g., therapeutic compositions, comprising one or more piRNA, e.g., exosome-derived piRNAs, and a pharmaceutically acceptable excipient. The present compositions find use in the treatment of a condition requiring tissue repair and/or regeneration, e.g., treatment of ischemic injury and/or tissue fibrosis. In some embodiments, the compositions are cell free and/or exosome-free compositions. In some embodiments, an exosome-free composition is substantially or essentially free of exosomes or extracellular vesicles. In some embodiments, an exosome-free composition does not include any exosomes or extracellular vesicles, or includes exosomes or extracellular vesicles in an amount that is insufficient to provide a detectable functional effect (e.g., when the composition is administered to a subject as provided herein). In some embodiments, a cell-free composition is substantially or essentially free of cells. In some embodiments, a cell-free composition does not include any cells, or includes cells in an amount that is insufficient to provide a detectable functional effect (e.g., when the composition is administered to a subject as provided herein). In several embodiments, the compositions comprise, consist of, or consist essentially of one or more exosome-derived RNA (e.g., piRNAs and/or miRNA), and a pharmaceutically acceptable excipient. In some embodiments, the composition comprises nucleic acids, proteins, or combinations thereof. The RNA, in several embodiments, comprises one or more of messenger RNA, snRNA, saRNA, miRNA, piRNA, and combinations thereof. In some embodiments, the exosome-derived RNA includes fibroblast-derived exosomal piRNA, e.g., piRNA derived from exosomes from engineered fibroblasts. In some embodiments, the exosome-derived RNA includes CDC-derived exosomal piRNA, e.g., piRNA derived from exosomes from immortalized CDCs. In several embodiments, the piRNA includes one or more of hsa_piR_016659, hsa_piR_016658, hsa_piR_001040, hsa_piR_007424, hsa_piR_008488, hsa_piR_018292, hsa_piR_013624, hsa_piR_019324, and hsa_piR_020548, e.g., as shown in FIG. 16. In some embodiments, the piRNA is hsa_piR_016659. In several embodiments, the piRNA includes one or more of piR-20450, piR-20548, piR-16735, piR-01184, piR-20786, piR-00805, piR-04153, piR-18570, piR-16677, and piR-17716. In several embodiments, the compositions comprise, consist of, or consist essentially of a synthetic piRNA and a pharmaceutically acceptable carrier. In some such embodiments, the synthetic piRNA comprises one or more of hsa_piR_016659, hsa_piR_016658, hsa_piR_001040, hsa_piR_007424, hsa_piR_008488, hsa_piR_018292, hsa_piR_013624, hsa_piR_019324, and hsa_piR_020548, e.g., as shown in FIG. 16. In some such embodiments, the synthetic piRNA is hsa_piR_016659. In several embodiments, the compositions comprise, consist of, or consist essentially of a synthetic piRNA and a pharmaceutically acceptable carrier. In several embodiments, the miRNA comprises one or more of miR-183-5p, miR-182-5p, miR-19a-3p, miR-92a-3p, miR-17-5p, miR-126-3p, and miR-510-3p, e.g., as shown in Table 1 below. In several embodiments, the miRNA comprises one or more of miR-92a, miR-182, miR-183, miR-19a, miR-26a, miR27-a, let-7e, mir-19b, miR-125b, mir-27b, let-7a, let-7c, miR-140-3p, miR-125a-5p, miR-150, miR-155, mir-210, let-7b, miR-24, miR-423-5p, miR-22, let-7f, miR-146a, and combinations thereof.

TABLE 1

| miRNA | Sequence | SEQ ID NO: |
|---|---|---|
| miR-183-5p | UAUGGCACUGGUAGAAUUCACU | 19 |
| miR-182-5p | UUUGGCAAUGGUAGAACUCACACU | 20 |
| miR-19a-3p | UGUGCAAAUCUAUGCAAAACUGA | 21 |
| miR-92a-3p | UAUUGCACUUGUCCCGGCCUGU | 22 |
| miR-17-5p | CAAAGUGCUUACAGUGCAGGUAG | 23 |
| miR-126-3p | UCGUACCGUGAGUAAUAAUGCG | 24 |
| miR-510-3p | AUUGAAACCUCUAAGAGUGGA | 25 |

In several embodiments, the compositions comprise a plurality of piRNA derived from a variety of cell types (e.g., piRNA isolated from a population of exosomes derived from a first and a second type of “parent cell”). As discussed above, in several embodiments, the compositions disclosed herein may be used alone, or in conjunction with one or more adjunct therapeutic modalities (e.g., pharmaceutical, cell therapy, gene therapy, protein therapy, surgery, etc.).

An RNA of the present disclosure (e.g., piRNA, miRNA) can be chemically modified at one or more positions along the nucleic acid. In some embodiments, the piRNA is chemically modified at one or more positions. In some embodiments, the miRNA is chemically modified at one or more positions. In some embodiments, the RNA includes one or more chemically modified nucleotides. A nucleotide can have any suitable chemical modification. In some embodiments, chemical modification of the RNA (e.g., piRNA, miRNA) increases in vitro and/or in vivo stability of the RNA. In some embodiments, chemical modification of the RNA (e.g., piRNA, miRNA) increases in vivo activity of the RNA.

In some embodiments, the RNA, e.g., piRNA or miRNA, contains from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

In some embodiments, the RNA, e.g., piRNA or miRNA, contains at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the RNA may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). In some embodiments, at least 5%, at least 10%, at least 25%, at least 50%, at least 80%, at least 90% or 100% of the cytosine in the nucleic acid is replaced with a modified cytosine (e.g., a 5-substituted cytosine).

Pharmaceutically acceptable excipients include, but not limited to, saline, aqueous buffer solutions, solvents and/or dispersion media. Some non-limiting examples of materials which can serve as pharmaceutically acceptable excipients include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. In some embodiments, the excipient inhibits the degradation of the active agent, e.g., piRNA.

In some embodiments, the composition is in a parenteral dose form. In some embodiments, parenteral dosage forms is sterile or capable of being sterilized before administering to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration to a subject. Suitable excipients that can be used to provide parenteral dosage forms of exosome-derived piRNA include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

In several embodiments, exosomes are formulated in a dosage form suitable for administration to a subject, e.g., intratracheal administration to a subject. In some embodiments, exosomes are formulated with a pharmaceutically acceptable excipient, as described herein. In some embodiments, exosomes are formulated for inhalation or nebulization, using any suitable option. In some embodiments, the exosomes are aerosolized.

Advantageously, in several embodiments, the exosomes comprise synthetic membrane bound particles (e.g., exosome surrogates), which depending on the embodiment, are configured to a specific range of diameters. In such embodiments, the diameter of the exosome surrogates is tailored for a particular application (e.g., target site or route of delivery). In still additional embodiments, the exosome surrogates are labeled or modified to enhance trafficking to a particular site or region post-administration.

In several embodiments, exosomes are obtained via centrifugation of the regenerative cells. In several embodiments, ultracentrifugation is used. However, in several embodiments, ultracentrifugation is not used. In several embodiments, exosomes are obtained via size-exclusion filtration of the regenerative cells. As disclosed above, in some embodiments, synthetic exosomes are generated, which can be isolated by similar mechanisms as those above.

Also provided herein are kits for treating a condition requiring tissue repair and/or regeneration (e.g., cardiac ischemic injury, pulmonary fibrosis), wherein the kit includes one or more exosome-derived piRNA species, as described herein, or a composition of the present disclosure. In some embodiments, the kit includes a pharmaceutically acceptable excipient, as described herein. Kits can include one or more containers (e.g., vials, ampoules, test tubes, flasks or bottles) for holding one or more components of the kits. The kits may further include instructions for using the kit to treat a condition requiring tissue repair and/or regeneration (e.g., cardiac ischemic injury, pulmonary fibrosis). The information and instructions may be in the form of words, pictures, or both, and the like.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### EXAMPLES

### Example 1

This non-limiting example illustrates detection and isolation of PIWI-interacting RNA (piRNA) from immortalized CDCs (imCDCs) and imCDC-derived extracellular vesicles (EVs)/exosomes.

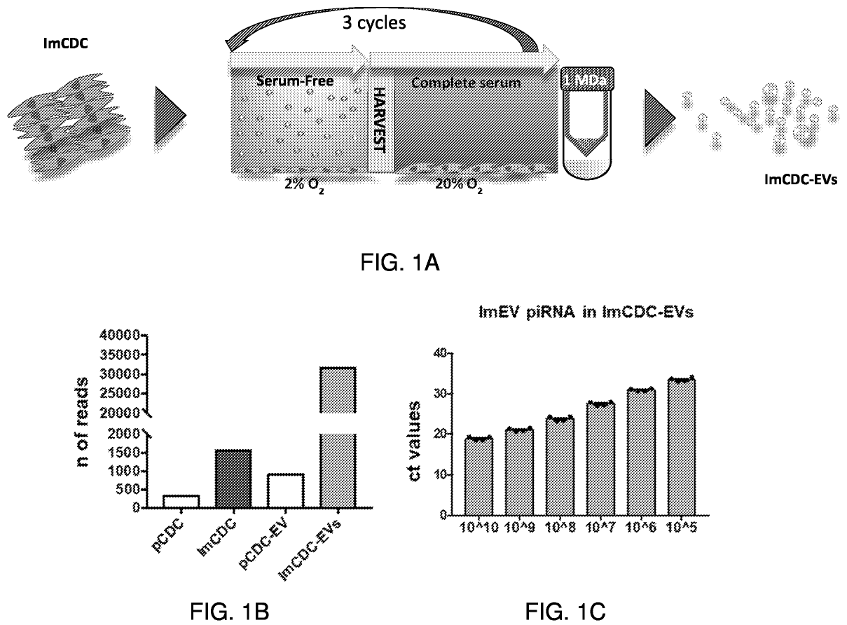

Extracellular Vesicles (EVs) were harvested from CDCs using a hypoxic cycling method used previously and as depicted in FIG. 1A. Briefly, cells were grown to confluence at 20% O₂/5% CO₂ at 37° C., and then cells were serum-free at 2% O₂/5% CO₂ at 37° C. about 24 hours. Conditioned media was collected and filtered through 0.45 μm filter to remove apoptotic bodies and cellular debris and frozen for later use at −80° C. The cells were cycled through 2% O₂/5% CO₂ conditions for three times, recovering in 20% O₂/5% CO₂ with complete serum for 48 hrs between exposure to hypoxic conditions. EVs were purified using centrifugal ultrafiltration with a 1000 KDa molecular weight cutoff filter. Fractions were analyzed in terms of particle size, number, and concentration and piRNA content.

The piRNA content of primary CDCs (pCDCs), imCDCs, pCDC-EVs and imCDC-EVs were analyzed using small-RNA sequence analysis. FIG. 1B shows piRNA was enriched in EVs compared to the cells from which they were derived. Further, immortalized CDCs and EVs isolated therefrom were enriched for piRNA compared to the non-immortalized CDCs and EVs derived therefrom. Thus, imCDCs show a different piRNA composition compared to primary CDC. ImEV-Pi (hsa_piR_016659) was identified as one of the most highly-expressed non-coding RNAs (the number of reads were 35× higher in imCDC-EV compared to CDC-EV) (FIG. 1B). As shown in FIG. 1C, the amount of piRNA correlated with the number of EVs.

These results show imCDCs are enriched for piRNA compared to primary CDCs. In some embodiments, EVs are enriched for piRNA. In some embodiments, imCDC-EVs are enriched for piRNA compared to pCDC-EVs.

### Example 2

This non-limiting example illustrates the in vivo cardioprotective effect of piRNA derived from imCDC-EV in myocardial ischemia/reperfusion (I/R) injury.

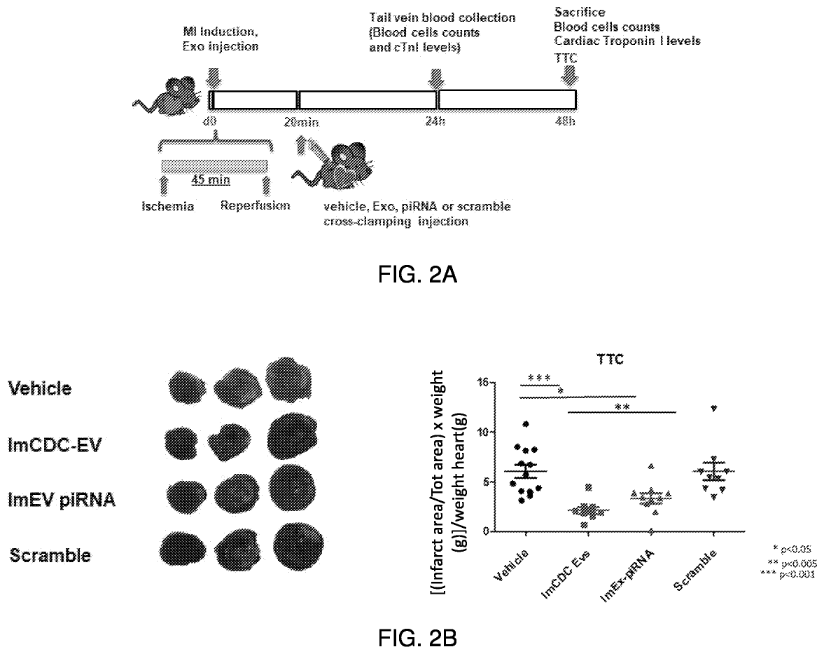

To investigate the role of piRNA derived from imCDC-EVs, a rat model of myocardial ischemia/reperfusion was used. FIG. 2A shows a schematic diagram of the experimental protocol. At day 0, myocardial infarction was surgically induced in 8-10 week-old Wistar-Kyoto female rats for 45 minutes, after which reperfusion was carried out. 20 minutes after reperfusion, vehicle, imCDC-EVs (10¹⁰ particles), imCDC-EV piRNA (hsa_piR_016659; 400 ng) or scramble RNA was administered intramyocardially with cross-clamping injection. 24 hours after ischemia/reperfusion tail vein blood was collected for blood cell counts and measurement of cardiac troponin I (cTnI) levels. After 48 hours, animals were sacrificed for blood cell counts, cTnI measurement and triphenyltetrazolium chloride (TTC) staining to measure scar size.

Animals administered imCDC-EV showed reduced scar size (infarct size) compared to vehicle based on TTC staining (FIG. 2B). Administration of imCDC-EV piRNA also showed reduced infarct size compared to vehicle. Infarct size was smaller in imCDC-EV treated animals compared to animals treated with imCDC-EV piRNA. In contrast, animals administered scrambled RNA showed no change in infarct size compared to vehicle-treated animals, while there was a trend in reduction of infarct size in animals administered imCDC-EV piRNA compared to animals administered scramble RNA.

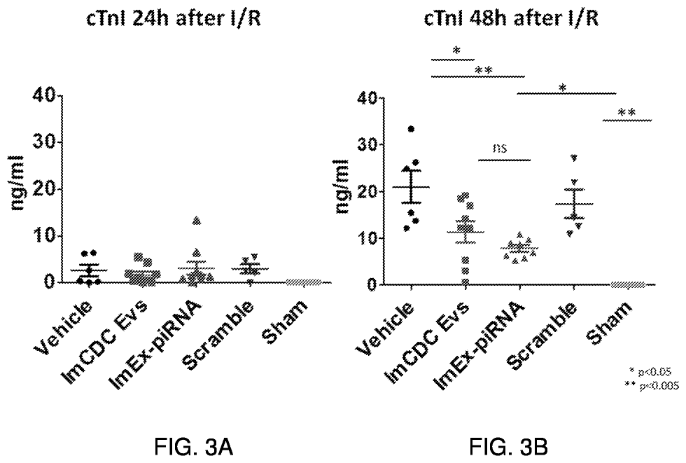

Cardiac troponin I (cTnI) in peripheral blood was used to measure the therapeutic effect of imCDC-EV piRNA after myocardial ischemia/reperfusion. At 24 hours, cTnI levels were low for all treatment groups (FIG. 3A). At 48 hours, cTnI levels increased in animals treated with vehicle as well as scramble RNA-treated animals (FIG. 3B). Animals administered imCDC-EV piRNA showed lower levels of cTnI compared to vehicle-treated animals. The level of cTnI in imCDC-EV piRNA-treated animals were similar to the level in animals administered imCDC-EVs. cTnI levels showed a lower trend in imCDC-EV piRNA-treated animals compared to scramble RNA-treated animals.

These results show imCDC-EV piRNA recapitulated the therapeutic effect of imCDC-EVs following myocardial ischemia/reperfusion injury. In some embodiments, administering imCDC-EV piRNA (e.g., hsa_piR_016659) is effective to reduce myocardial infarct size after cardiac injury. In some embodiments, administering imCDC-EV piRNA (e.g., hsa_piR_016659) reduces blood cTnI levels after cardiac injury.

### Example 3

This non-limiting example illustrates imCDC-EVs and imCDC-EV piRNA change peripheral monocyte population dynamics following myocardial ischemia/reperfusion injury.

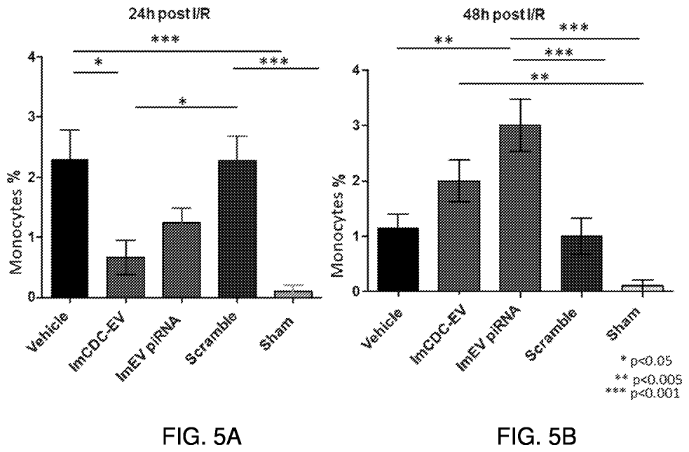

The effect of administering imCDC-EV piRNA (hsa_piR_016659) on changes in the fraction of monocytes in peripheral blood of animals after myocardial ischemia/reperfusion injury was studied. At 24 hours after myocardial ischemia/reperfusion, vehicle-treated and scramble RNA-treated animals showed an increase in monocytes compared to sham operated animals (FIG. 5A). In contrast, animals treated with imCDC-EVs had a lower percentage of monocytes compared to vehicle-treated and scramble RNA-treated animals. imCDC-EV piRNA-treated animals showed a similar trend of lower percentage of monocytes compared to vehicle-treated and scramble RNA-treated animals.

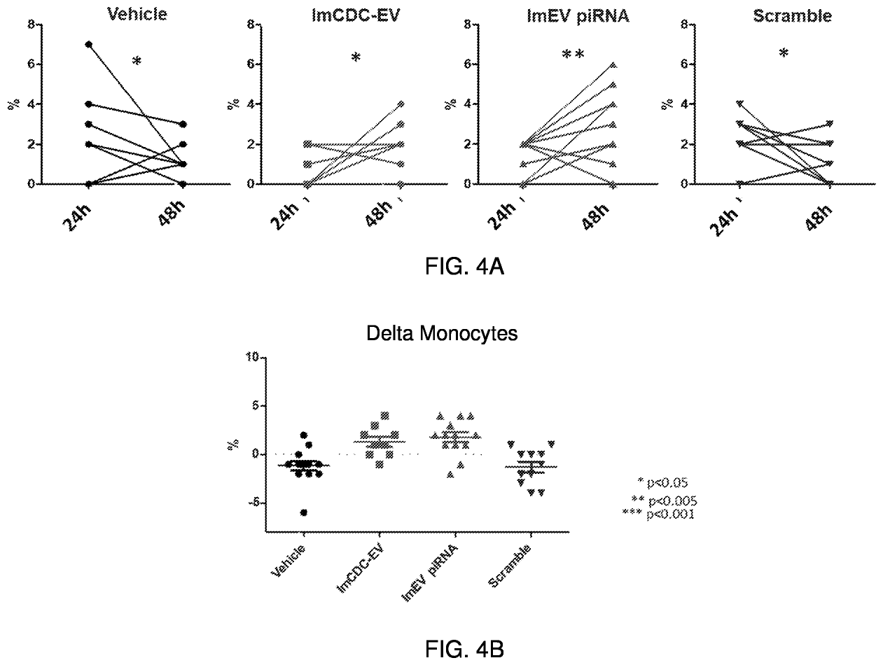

The percentage of monocytes in peripheral blood changed over the next 24 hours. In animals treated with vehicle or scramble RNA, the percentage of monocytes declined from 24 hours to 48 hours post-myocardial ischemia/reperfusion (FIGS. 4A, 4B). In contrast, in imCDC-EV-treated and imCDC-EV piRNA-treated animals, the percentage of monocytes increased.

At 48 hours after myocardial ischemia/reperfusion, imCDC-EV piRNA-treated animals had a higher percentage of monocytes than vehicle-treated and scramble RNA-treated animals (FIG. 5B). ImCDC-EV-treated animals showed a trend for higher percentage of monocytes compared to vehicle-treated and scramble RNA-treated animals. In contrast, imCDC EV piRNA had only minimal effect on neutrophil counts profile in blood.

These results show imCDC-EV and imCDC-EV piRNA administration can change monocyte population dynamics in peripheral blood after myocardial ischemia/reperfusion injury. Monocytes may be a target of imCDC-EV and imCDC-EV piRNA. In some embodiments, imCDC-EV and imCDC-EV piRNA (e.g., hsa_piR_016659) change monocyte composition in peripheral blood. In some embodiments, imCDC-EV piRNA (e.g., hsa_piR_016659) administration suppresses an increase in monocytes in peripheral blood 24 hours after myocardial ischemia/reperfusion injury. In some embodiments, imCDC-EV piRNA (e.g., hsa_piR_016659) administration delays an increase in monocytes in peripheral blood during 24 to 48 hours after myocardial ischemia/reperfusion injury.

### Example 4

This non-limiting example shows increased in vitro survival, proliferation and migration of primary macrophages cultured in the presence of imCDC-EV and imCDC-EV piRNA.

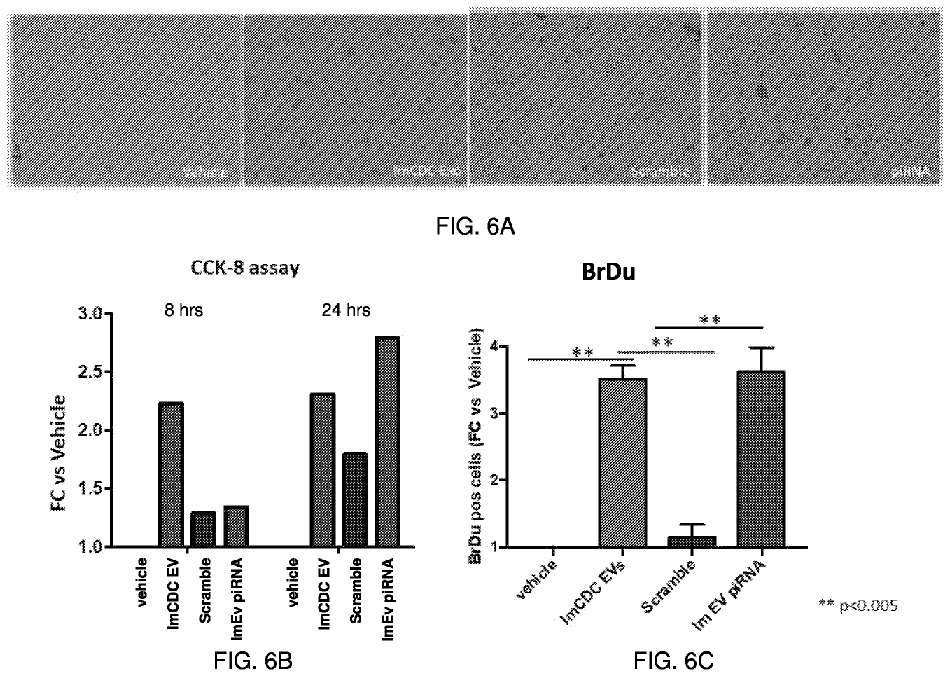

The effect of imCDC-EV and imCDC-EV piRNA (hsa_piR_016659) on monocytes were studied in vitro. Naïve (M0) macrophages derived from bone marrow-derived macrophages (BMDM) were cultured for 24 hours in the presence of imCDC-EV, imCDC-EV piRNA or scramble RNA, and tested for cell viability and proliferation. FIG. 6A shows images of the BMDM-derived M0 macrophages after the 24 hour culture. ImCDC-EV-treated cells showed an over 2-fold increase in cell viability compared to vehicle control at 8 and 24 hours of culture (FIG. 6B), and exhibited more than 3-fold increase in proliferation at 24 hours (FIG. 6C). Cells cultured with imCDC-EV piRNA also showed enhanced viability and proliferation compared to vehicle control at 24 hours. In contrast, cells cultured with scramble RNA showed cell proliferation comparable to that of vehicle control. Scramble RNA-treated cells did show enhanced survival compared to vehicle at 24 hours, but at a level less than imCDC-EV or imCDC-EV piRNA treated cells.

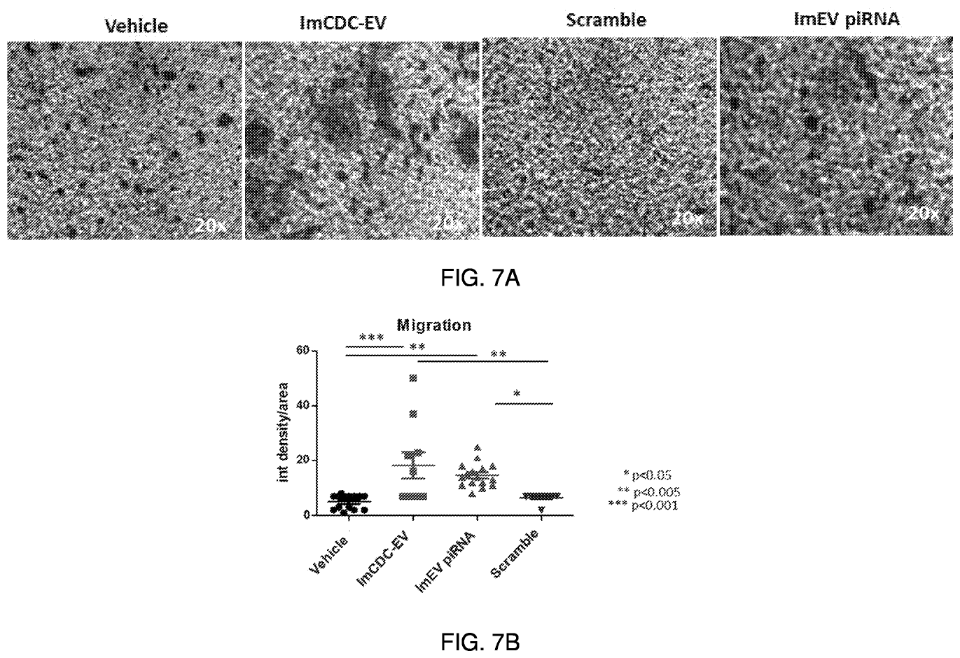

Migration of macrophages treated with imCDC-EV, imCDC-EV piRNA or scramble RNA were tested in vitro. FIG. 7A shows images of the BMDM-derived M0 macrophages in polycarbonate inserts stained with Crystal violet. Macrophages grown with imCDC-EV or imCDC-EV piRNA showed greater migration compared to vehicle- and scramble RNA-treated cells (FIG. 7B).

These results show imCDC-EV and/or imCDC-EV piRNA can act directly on monocytes to promote survival, proliferation and migration of the cells.

### Example 5

This non-limiting example shows changes in macrophages transcriptome induced imCDC-EV piRNA.

In vitro, bone marrow derived-macrophages (BMDM) were exposed to imCDC-EV, imCDC-EV piRNA (hsa_piR_016659) and control, as in Example 4. Transcriptome profile and activated pathways were then assessed. ImCDC-EV piRNA-conditioned BMDM exhibited a different transcriptomic profile compared with control, with upregulation of pathways involved in the inflammatory response, cell death, and cell-to cell signaling.

These results show macrophages may be a target of imCDC-EV and imCDC-EV piRNA. In some embodiments, imCDC-EV and/or imCDC-EV piRNA (e.g., hsa_piR_016659) alters mRNA expression profile in macrophages.

### Example 6

This non-limiting example illustrates identification of anti-fibrotic mediators in ASTEX (extracellular vesicles/exosomes from Activated-Specialized Tissue Effector Cells (ASTECs)).

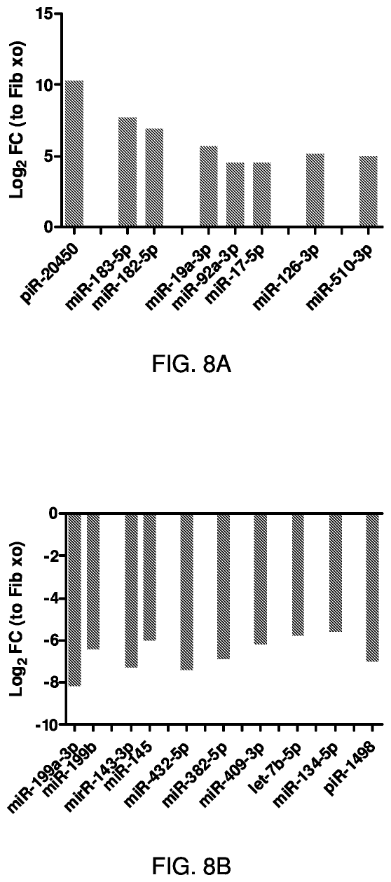

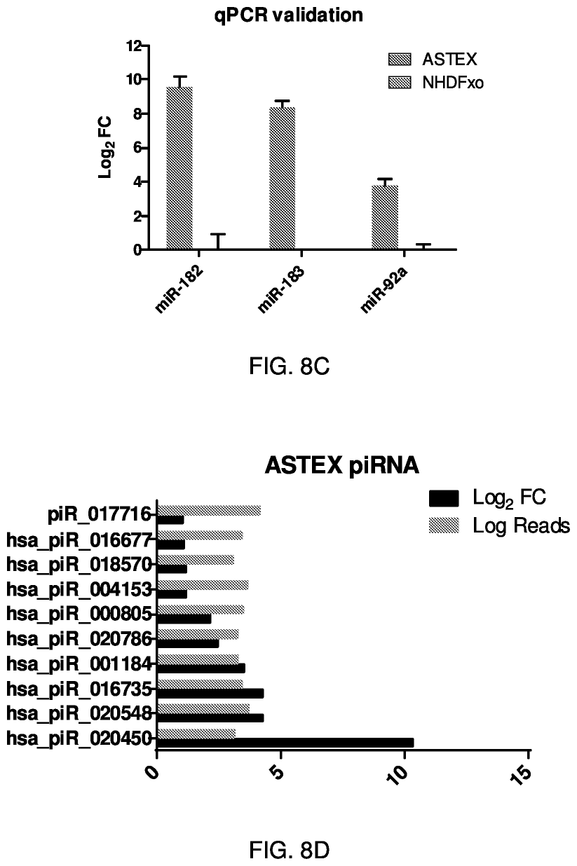

RNA was isolated from extracellular vesicles (EVs) produced by ASTECs that were cultured using a 15-day serum starvation method. Sequencing of the isolated RNA revealed enriched expression of miRNA and piRNA species, including those implicated as anti-fibrotic mediators, compared to EVs from unmodified normal human dermal skin fibroblasts (FIGS. 8A, 8D). In particular, the miR-183 family and miR-17-92 family of miRNAs, which are known to target and inhibit pathological drivers of idiopathic pulmonary fibrosis (IPF) were enriched. Enrichment of miR-182, miR-183 and miR-92a was confirmed by qPCR (FIG. 8C). In contrast, ASTEX were depleted for a set of miRNA and piRNA compared to EVs from unmodified normal human dermal skin fibroblasts (FIG. 8B).

These results show ASTEX can be enriched for anti-fibrotic mediators including miRNA species that target pathological drivers of IPF. In some embodiments, ASTEX are enriched for miR-182, miR-183 and miR-92a. In some embodiments, ASTEX are enriched for piR-20450.

### Example 7

This non-limiting example illustrates the therapeutic effect of ASTEX in idiopathic lung fibrosis.

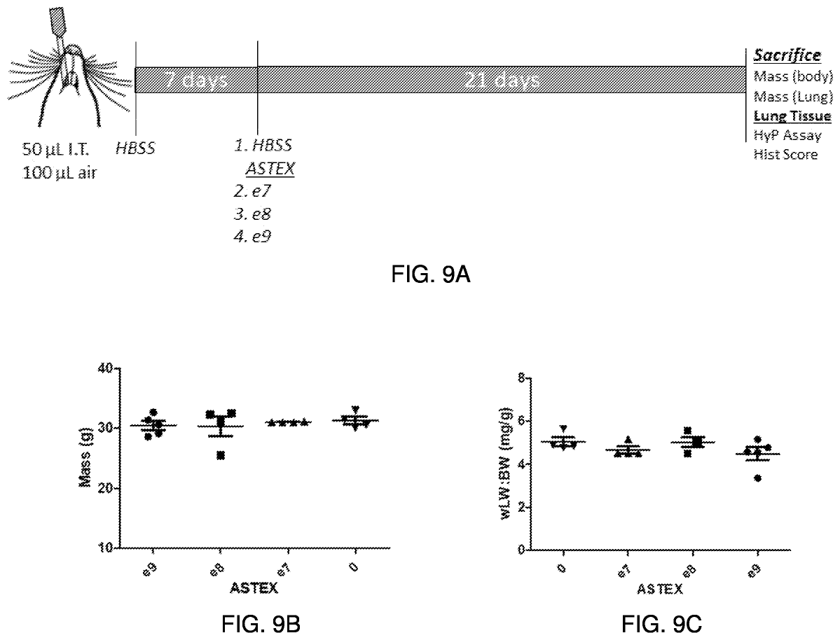

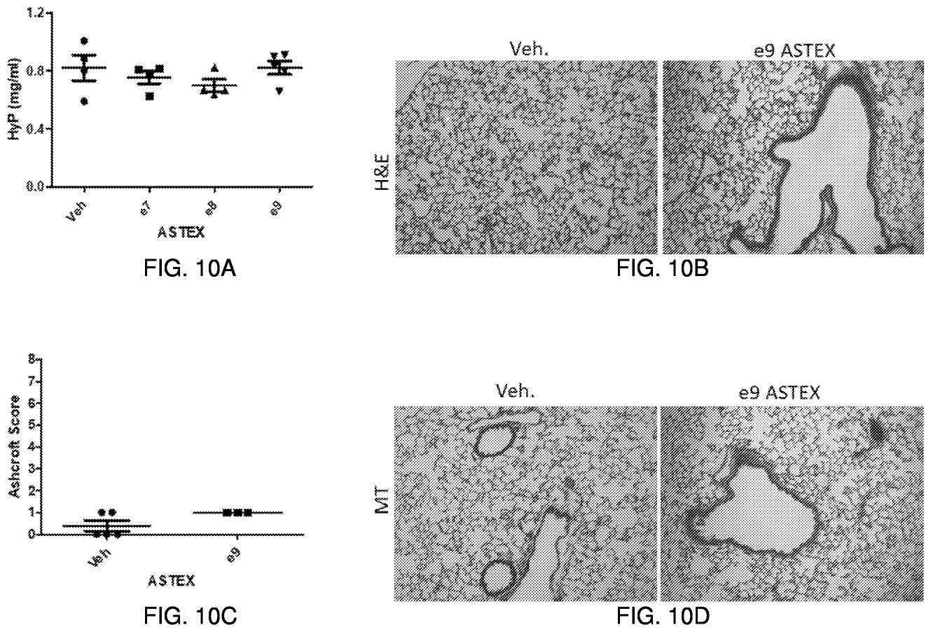

A mouse model of idiopathic lung fibrosis was used to test the therapeutic effect of ASTEX. First, a dose tolerance study for ASTEX was performed, as shown schematically in FIG. 9A. At the start of the study, 50 μL of saline solution (HBSS) was administered intrathecally with 100 μL of air. 7 days later, ASTEX was administered intrathecally at doses of 10⁷, 10⁸, and 10⁹ particles. 21 days after administration of ASTEX, body mass, lung mass, and lung hydroxyproline levels (HyP) were measured, and histological staining of alveolar tissue was performed. Animals administered ASTEX at each dosage retained body weight and did not show lung edema relative to vehicle control (FIG. 9B), indicating ASTEX were well tolerated. Lung hydroxyproline levels at all dosages were comparable to vehicle control (FIG. 10A), indicating lack of fibrosis animals administered ASTEX. Histological characterization of alveolar tissue also showed lack of fibrosis in animals administered 10⁹ ASTEX. Ashcroft score of alveolar tissue from animals administered ASTEX was comparable to vehicle control (FIG. 10B). H&E staining showed no infiltrating leukocytes in alveolar tissue from animals administered ASTEX (FIG. 10C), nor was there evidence of fibrosis based on Masson's trichome staining (FIG. 10D).

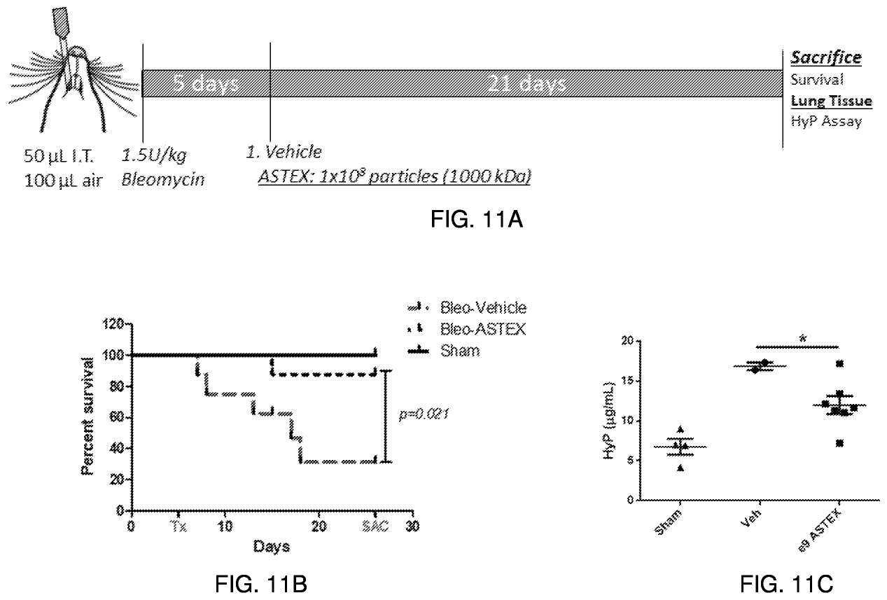

After confirming ASTEX were well tolerated by healthy animals, the EVs were administered to animals in which pulmonary fibrosis was induced by bleomycin (FIG. 11A). 1×10⁸ particles of ASTEX (1000 kDa) were administered intrathecally 5 days after the animals were administered bleomycin (FIG. 11A).

Bleomycin-treated animals that were administered ASTEX showed improved survival compared to vehicle-treated animals (FIG. 11B). Further, ASTEX administration reduced lung hydroxyproline levels compared to vehicle-treated animals (FIG. 11C).

These results show ASTEX have a therapeutic effect in treating idiopathic lung fibrosis (IPF). In some embodiments, ASTEX reduces or delays mortality from IPF. In some embodiments, ASTEX reduces or delays lung fibrosis in IPF.

### Example 8

This non-limiting example illustrates the effect of ASTEX on lung fibroblast transdifferentiation in vitro.

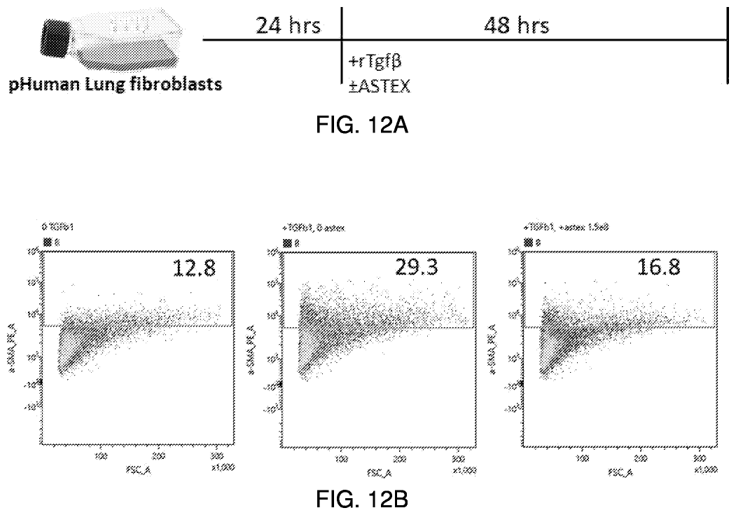

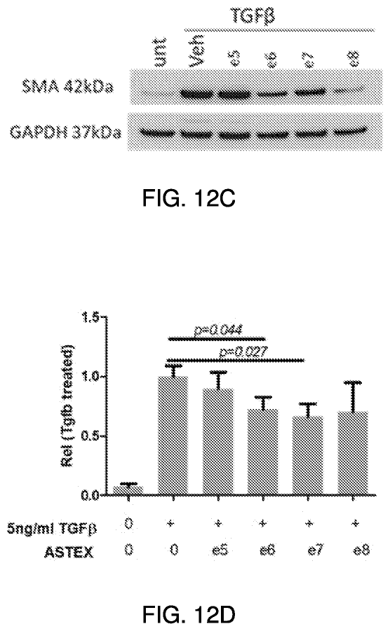

To understand the mechanism underlying the therapeutic effect of ASTEX in IPF, the effect of ASTEX on primary human lung fibroblasts was studied in vitro. Cells were treated with TGFβ to mimic injury promoting lung fibroblast transdifferentiation into myofibroblast (FIG. 12A). Cells were concurrently treated with either ASTEX or vehicle solution. ASTEX treatment attenuated TGFβ-induced upregulation of a smooth muscle actin (α-SMA) compared to vehicle-treated cells (FIGS. 12B-12D).

These results show ASTEX can reduce injury-induced transdifferentiation of lung fibroblast into myofibroblast. In some embodiments, ASTEX attenuates upregulation of α-SMA induced by TGFβ in lung fibroblasts.

### Example 9

This non-limiting example illustrates a method of treating idiopathic pulmonary fibrosis by administering piRNA from ASTEX.

piRNA is isolated from ASTEX. Animals are exposed intratracheally to bleomycin to induce pulmonary fibrosis. The ASTEX-derived piRNA is administered intratracheally to the animals 5 days after bleomycin exposure. Animals are observed for survival, and body weight is measured over 21 days after administration of piRNA. Hydroxyproline levels in lung tissue are measured to estimate the level of lung fibrosis.

### Example 10

This non-limiting example illustrates shuttling of imCDC-EV piRNA between the cytoplasm and nucleus.

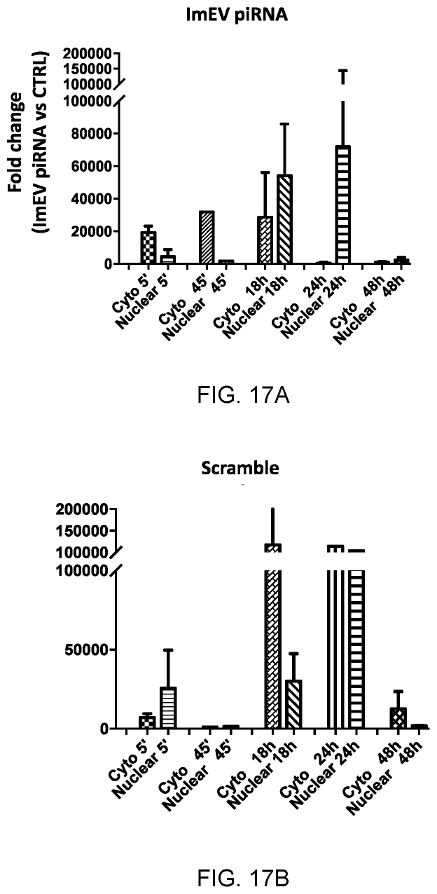

BMDM-derived M0 macrophages were grown in the presence of imCDC-EV piRNA (hsa_piR_016659) or vehicle control and the subcellular localization of piRNA as a fold change over control level was measured at different time points (see Example 4). piRNA was initially more abundant in the cytoplasm than in the nucleus starting from 5 minutes after start of culturing, and was predominantly in the cytoplasm at 45 minutes (FIG. 17A). At 18 hours, the piRNA was found in the cytoplasm and the nuclear compartment, and by 24 hours, piRNA was predominantly in the nucleus, while very little remained in the cytoplasm (FIG. 17A). By 48 hours, piRNA was not found at any significant level in the cytoplasm or the nucleus (FIG. 17A). In contrast, scramble RNA was present in the cytoplasm and nucleus at 5′, 18 hours and at 24 hours, and was mostly eliminated in both compartments by 48 hours (FIG. 17B). This result shows that imCDC-EV piRNA (hsa_piR_016659) preferentially accumulates in the nucleus where it may regulate gene expression.

### Example 11

This non-limiting example illustrates increase in global methylation in primary macrophages treated with imCDC-EV piRNA.

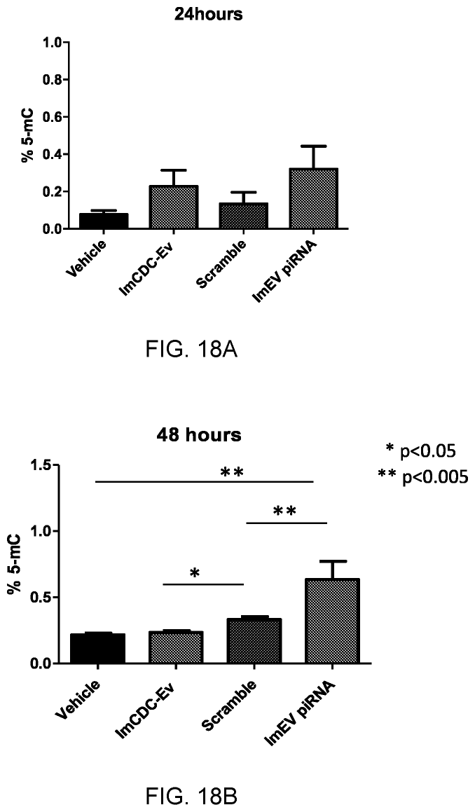

BMDM-derived M0 macrophages were grown in the presence of imCDC-EV, imCDC-EV piRNA (hsa_piR_016659), scramble RNA, or vehicle control and levels of global methylation was measured at 24 and 48 hours (see Example 4). At 24 hours, cells treated with imCDC-EV and imCDC-EV piRNA showed an increase in global methylation compared to vehicle control or scramble RNA (FIG. 18A). At 48 hours, cells treated with imCDC-EV had methylation levels comparable to vehicle control (FIG. 18B). In contrast, primary macrophages treated with imCDC-EV piRNA maintained an elevated level of global methylation compared to vehicle control or scramble RNA (FIG. 18B).

This result shows that imCDC-EV piRNA (hsa_piR_016659) can increase DNA methylation, which may contribute to regulation of gene expression.

In some embodiments, contacting primary macrophages with imCDC-EV and/or imCDC-EV piRNA (e.g., hsa_piR_016659) increases global methylation in the primary macrophages. In some embodiments, changes in global methylation level induced by contacting primary macrophages with imCDC-EV piRNA (e.g., hsa_piR_016659) are longer lasting than global methylation changes induced by imCDC-EV.

### Example 11

This non-limiting example shows a schematic diagram summarizing the effects of imCDC-EV piRNA (e.g., hsa_piR_016659) on primary macrophages, as shown in Examples 1-5, 10 and 11.

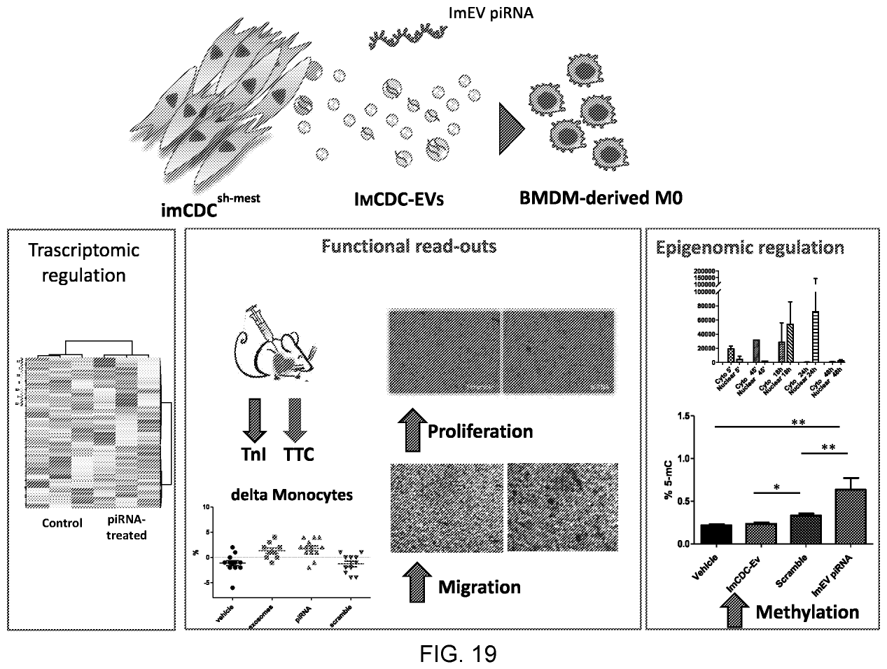

The data disclosed in Examples 1-5, 10 and 11 support a model of imCDC-EV and/or imCDC-EV piRNA (hsa_piR_016659) cardioprotection in myocardial ischemia/reperfusion (I/R) injury through their action on BMDM-derived M0 macrophages (FIG. 19, upper panel). BMDM cultured with imCDC-EVs and/or imCDC-EV piRNA (hsa_piR_016659) exhibited altered transcriptional profile that indicated upregulation of pathways involved in the inflammatory response, cell death, and cell-to cell signaling (Example 5; FIG. 19, lower left panel). imCDC-EV piRNA (hsa_piR_016659) was shuttled to preferentially to the nucleus and increased global DNA methylation (Example 10; FIG. 19, lower right panel). When administered to animals in a model of myocardial ischemia/reperfusion injury, imCDC-EVs and/or imCDC-EV piRNA (hsa_piR_016659) reduced infarct size and cTnI levels in the periphery (Example 2; FIG. 19, lower center panel). Peripheral monocyte population dynamics following myocardial ischemia/reperfusion injury was also altered by administration of imCDC-EVs and/or imCDC-EV piRNA (Example 3; FIG. 19, lower center panel). In vitro, imCDC-EVs and/or imCDC-EV piRNA (hsa_piR_016659) increased survival, proliferation and migration of primary macrophages (Example 4; FIG. 19, lower center panel).

Although the foregoing has been described in some detail by way of illustrations and examples for purposes of clarity and understanding, it will be understood by those of skill in the art that modifications can be made without departing from the spirit of the present disclosure. Therefore, it should be understood that the forms disclosed herein are illustrative only and are not intended to limit the scope of the present disclosure, but rather to also cover all modification and alternatives coming with the true scope and spirit of the embodiments of the present disclosure.

It is contemplated that various combinations or subcombinations of the specific features and aspects of the embodiments disclosed above may be made. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed subject matter. Thus, it is intended that the scope of the present disclosure should not be limited by the particular disclosed embodiments described above. Moreover, while the disclosed subject matter is susceptible to various modifications, and alternative forms, specific examples thereof have been shown in the drawings and are herein described in detail. It should be understood, however, that the present disclosure is not to be limited to the particular forms or methods disclosed, but is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering an effective (or therapeutically effective) amount of exosome-derived PIWI-interacting RNA (piRNA)” include “instructing the administration of an effective (or therapeutically effective) amount of exosome-derived PIWI-interacting RNA (piRNA).” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% homologous includes 96%, 97%, 98%, 99%, and 100% homologous to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like.

The indefinite article “a” or “an” does not exclude a plurality. The term “about” as used herein to, for example, define the values and ranges of molecular weights means that the indicated values and/or range limits can vary within ±20%, e.g., within ±10%, including within ±5%. The use of “about” before a number includes the number itself. For example, “about 5” provides express support for “5.” Numbers provided in ranges include overlapping ranges and integers in between; for example a range of 1-4 and 5-7 includes for example, 1-7, 1-6, 1-5, 2-5, 2-7, 4-7, 1, 2, 3, 4, 5, 6 and 7.

What is claimed is:

1. An exosome-free method of treating ischemic cardiac muscle injury, comprising:
identifying a subject in need of treating an ischemic cardiac muscle injury; and
administering to the subject a therapeutically effective amount of a PIWI-interacting RNA (piRNA) comprising a nucleotide sequence of hsa_piR_016659 (SEQ ID NO: 1), to thereby treat the ischemic cardiac muscle injury.

2. The method of claim 1, wherein the piRNA consists of the nucleotide sequence of hsa_piR_016659.

3. The method of claim 1, wherein the ischemic cardiac muscle injury comprises ischemic/reperfusion injury.

4. The method of claim 1, wherein the ischemic cardiac muscle injury comprises cardiac muscle fibrosis.

5. The method of claim 1, wherein the subject has suffered a myocardial infarction.

6. The method of claim 1, wherein the therapeutically effective amount of the piRNA is administered about 10 minutes to about 2 hours after the ischemic cardiac muscle injury.

7. The method of claim 1, wherein the therapeutically effective amount comprises from about 80 ng to about 5 mg of the piRNA.

8. The method of claim 1, wherein the piRNA comprises one or more chemically modified nucleotides.

9. An exosome-free method of treating ischemic cardiac muscle injury, comprising:
identifying a subject in need of treatment for an ischemic cardiac muscle injury; and
administering to the subject a therapeutically effective amount of an exosome-derived PIWI-interacting RNA (piRNA), wherein the therapeutically effective amount comprises from about 80 ng to about 5 mg of the piRNA, to thereby treat the ischemic cardiac muscle injury.

10. The method of claim 9, wherein the exosome-derived piRNA comprises CDC (cardiosphere-derived cells)-derived exosomal piRNA.

11. The method of claim 10, wherein the exosome-derived piRNA comprises one or more of: hsa_piR_016659 (SEQ ID NO: 1), hsa_piR_016658 (SEQ ID NO: 2), hsa_piR_001040 (SEQ ID NO: 3), hsa_piR_007424 (SEQ ID NO: 4), hsa_piR_008488 (SEQ ID NO: 5), hsa_piR_018292 (SEQ ID NO: 6), hsa_piR_013624 (SEQ ID NO: 7), hsa_piR_019324 (SEQ ID NO: 8), and hsa_piR_020548 (SEQ ID NO: 9).

12. The method of claim 11, wherein the exosome-derived piRNA is hsa_piR_016659.

13. The method of claim 10, wherein the ischemic cardiac muscle injury comprises ischemic/reperfusion injury.

14. The method of claim 10, wherein the ischemic cardiac muscle injury comprises cardiac muscle fibrosis.

15. The method of claim 10, wherein the subject has suffered a myocardial infarction.

16. The method of claim 10, wherein the therapeutically effective amount of exosome-derived piRNA is administered about 10 minutes to about 2 hours after the ischemic cardiac muscle injury.

17. An exosome-free method of treating a condition requiring tissue repair and/or regeneration, comprising:
identifying a subject having a condition requiring tissue repair and/or regeneration; and
administering to the subject a therapeutically effective amount of exosome-derived PIWI-interacting RNA (piRNA), wherein the therapeutically effective amount comprises from about 80 ng to about 5 mg of the piRNA, to thereby treat the condition requiring tissue repair and/or regeneration.

18. The method of claim 17, wherein the condition requiring tissue repair and/or regeneration comprises injury to muscle or lung tissue.

19. The method of claim 18, wherein the muscle tissue comprises cardiac or skeletal muscle.

20. The method of claim 18, wherein the condition is a condition that causes tissue fibrosis.

21. The method of claim 18, wherein the condition comprises ischemic cardiac muscle injury or pulmonary fibrosis.

22. The method of claim 18, wherein the exosome-derived piRNA comprises CDC (cardiosphere-derived cells)-derived exosomal piRNA or fibroblast-derived exosomal piRNA.

23. The method of claim 22, wherein the exosome-derived piRNA comprises one or more of: hsa_piR_016659, hsa_piR_016658, hsa_piR_001040, hsa_piR_007424, hsa_piR_008488, hsa_piR_018292, hsa_piR_013624, hsa_piR_019324, and hsa_piR_020548.

24. The method of claim 23, wherein the exosome-derived piRNA is hsa_piR_016659.

25. A cell-free method of treating a condition requiring tissue repair and/or regeneration, comprising:
identifying a subject having a condition requiring tissue repair and/or regeneration; and
administering to the subject a therapeutically effective amount of exosome-derived PIWI-interacting RNA (piRNA), to thereby treat the condition requiring tissue repair and/or regeneration,
wherein the exosome-derived piRNA comprises one or more of hsa_piR_016659 (SEQ ID NO: 1), hsa_piR_016658 (SEQ ID NO: 2), hsa_piR_001040 (SEQ ID NO: 3), hsa_piR_007424 (SEQ ID NO: 4), hsa_piR_008488 (SEQ ID NO: 5), hsa_piR_018292 (SEQ ID NO: 6), hsa_piR_013624 (SEQ ID NO: 7), hsa_piR_019324 (SEQ ID NO: 8), hsa_piR_020548 (SEQ ID NO: 9), piR-20450 (SEQ ID NO: 10), piR-16735 (SEQ ID NO: 11, piR-01184 (SEQ ID NO: 12), piR-20786 (SEQ ID NO: 13), piR-00805 (SEQ ID NO: 14, piR-04153 (SEQ ID NO: 15), piR-18570 (SEQ ID NO: 16), piR-16677 (SEQ ID NO: 17, and piR-17716 (SEQ ID NO: 18).

26. The method of claim 25, wherein administering comprises administering a therapeutically effective amount of exosomes, extracellular vesicles or liposomes comprising the exosome-derived piRNA, wherein the exosomes, extracellular vesicles or liposomes are enriched for the exosome-derived piRNA.

27. The method of claim 25, wherein the therapeutically effective amount comprises from about 80 ng to about 5 mg of the exo some-derived piRNA.

28. The method of claim 27, wherein the condition requiring tissue repair and/or regeneration comprises injury to muscle or lung tissue.

29. The method of claim 28, wherein the condition is a condition that causes tissue fibrosis.

30. The method of any one of claims 25-29, wherein the exosome-derived piRNA comprises fibroblast-derived exosomal piRNA or CDC (cardiosphere-derived cells)-derived exosomal piRNA.

31. The method of any one of claims 9-30, wherein the exosome-derived piRNA comprises one or more chemically modified nucleotides.

32. The method of any one of the preceding claims, wherein the piRNA is administered intravenously, intra-arterially, intramuscularly, intracardially, intramyocardially or intratracheally.

33. A cell-free method of treating pulmonary fibrosis, comprising:
identifying a subject having pulmonary fibrosis; and
administering to the subject a therapeutically effective amount of exosome-derived PIWI-interacting RNA (piRNA), therapeutic exosomes, and/or exosome-derived miRNA, to thereby treat the pulmonary fibrosis, wherein the exosomes are derived from engineered fibroblasts.

34. The method of claim 33, wherein the therapeutically effective amount of exosome-derived piRNA, therapeutic exosomes, and/or exosome-derived miRNA, is administered intratracheally.

35. The method of claim 33, wherein the therapeutically effective amount of the therapeutic exosomes comprises from about 10⁶ to about 10¹² particles.

36. A method of regulating tissue repair, comprising contacting a population of transdifferentiating fibroblasts with a therapeutically effective amount of exosome-derived PIWI-interacting RNA (piRNA), exosomes, and/or exosome-derived miRNA, to thereby suppress transdifferentiation of the fibroblasts into myofibroblasts, wherein the exosomes are derived from engineered fibroblasts.

37. The method of claim 36, wherein the therapeutically effective amount of exosomes comprises about 10⁶ to about 10¹² particles.

38. The method of claim 36, wherein the transdifferentiation is TGFβ-mediated transdifferentiation.

39. The method of claim 36, wherein the contacting is done in vitro.

40. The method of claim 39, wherein the therapeutically effective amount of piRNA comprises from about 1 nM to about 200 nM.

41. The method of claim 36, wherein the contacting comprises administering the exosomes to a subject.

42. The method of claim 36, wherein the transdifferentiating fibroblasts are lung fibroblasts.

43. The method of claim 42, wherein contacting comprises administering the exosome-derived piRNA, exosomes, and/or exosome-derived miRNA, to a subject intratracheally.

44. The method of claim 43, wherein the subject has pulmonary fibrosis.

45. The method of claim 36, wherein the contacting comprises contacting the population of transdifferentiating fibroblasts with a therapeutically effective amount of exosome-derived miRNA, wherein the exosome-derived miRNA comprises one or more of miR-183-5p (SEQ ID NO: 19), miR-182-5p (SEQ ID NO: 20), miR-19a-3p (SEQ ID NO: 21, miR-92a-3p (SEQ ID NO: 22), miR-17-5p (SEQ ID NO: 23), miR-126-3p (SEQ ID NO: 24, and miR-510-3p (SEQ ID NO: 25).

46. The method of claim 36, wherein the contacting comprises contacting the population of transdifferentiating fibroblasts with a therapeutically effective amount of exosome-derived piRNA, wherein the exosome-derived piRNA comprises one or more of piR-20450 (SEQ ID NO: 10), piR-20548 (SEQ ID NO: 9), piR-16735 (SEQ ID NO: 11), piR-01184 (SEQ ID NO: 12), piR-20786 (SEQ ID NO: 13), piR-00805 (SEQ ID NO: 14), piR-04153 (SEQ ID NO: 15), piR-18570 (SEQ ID NO: 16), piR-16677 (SEQ ID NO: 17), and piR-17716 (SEQ ID NO: 18).

47. The method of any one of the preceding claims, comprising isolating the piRNA from therapeutic exosomes.

48. The method of claim 47, wherein the therapeutic exosomes are CDC-derived exosomes or fibroblast-derived exosomes.

49. The method of any one of claims 33-48, comprising isolating the therapeutic exosomes from a population of therapeutic cells.

50. The method of claim 49, comprising generating the population of therapeutic cells from non-therapeutic cells.

51. The method of claim 50, wherein the non-therapeutic cells comprise fibroblasts or CDCs.

52. The method of claim 51, wherein the CDCs are immortalized CDCs.

53. The method of any one of claims 49-52, wherein the therapeutic cells are allogeneic.

54. The method of any one of claims 33-53, wherein the therapeutically effective amount of exosome-derived piRNA is from about 80 ng to about 500 μg.

55. The method of claim 54, wherein the therapeutically effective amount of exosome-derived piRNA is from about 100 ng to about 10 μg.

56. The method of any one of the preceding claims, wherein the therapeutically effective amount of the piRNA is an amount having a therapeutic effect equivalent to a therapeutic effect of administering from about 10⁹ to about 10¹² immortalized CDC-derived exosomes.

57. Use of exosome-derived PIWI-interacting RNA (piRNA) to treat ischemic cardiac injury in a subject in need thereof.

58. Use of exosome-derived PIWI-interacting RNA (piRNA) for the preparation of a medicament to treat ischemic cardiac injury in a subject in need thereof.

59. The use of exosome-derived piRNA of claims 57 and 58, wherein the exosome-derived piRNA comprises one or more of hsa_piR_016659, hsa_piR_016658, hsa_piR_001040, hsa_piR_007424, hsa_piR_008488, hsa_piR_018292, hsa_piR_013624, hsa_piR_019324, and hsa_piR_020548.

60. Use of therapeutic exosomes and/or exosome-derived PIWI-interacting RNA (piRNA) to treat pulmonary fibrosis in a subject in need thereof.

61. Use of therapeutic exosomes and/or exosome-derived PIWI-interacting RNA (piRNA) for the preparation of a medicament to treat pulmonary fibrosis in a subject in need thereof.

62. The use of therapeutic exosomes and/or exosome-derived piRNA of claim 60 or 61, wherein the therapeutic exosomes and/or exosome-derived piRNA comprises one or more of piR-20450, piR-20548, piR-16735, piR-01184, piR-20786, piR-00805, piR-04153, piR-18570, piR-16677, and piR-17716.

63. An exosome-free therapeutic composition for treatment of a condition requiring tissue repair and/or regeneration, comprising:
one or more exosome-derived piRNAs selected from hsa_piR_016659, hsa_piR_016658, hsa_piR_001040, hsa_piR_007424, hsa_piR_008488, hsa_piR_018292, hsa_piR_013624, hsa_piR_019324, and hsa_piR_020548; and
a pharmaceutically acceptable excipient.

64. The composition of claim 63, consisting essentially of the one or more exosome-derived piRNAs and the pharmaceutically acceptable excipient.

65. The composition of claim 63, wherein the one or more exosome-derived piRNAs is hsa_piR_016659.

66. The composition of claim 63, wherein the condition is a condition that causes tissue fibrosis.

67. The composition of claim 63, wherein the condition comprises ischemic cardiac muscle injury or pulmonary fibrosis.

68. The composition of any one of claims 63-67, wherein the one or more exosome-derived piRNAs comprises fibroblast-derived exosomal piRNA or CDC (cardiosphere-derived cells)-derived exosomal piRNA.
